(19) United States
(12) Patent Application Publication
(10) Pub. No.: US 2021/0251665 A1
(43) Pub. Date: Aug. 19, 2021

(54) SYSTEM FOR INFORMATIONAL MAGNETIC FEEDBACK IN ADJUSTABLE IMPLANTS

(71) Applicant: NuVasive Specialized Orthopedics, Inc., San Diego, CA (US)

(72) Inventor: Shanbao Cheng, Irvine, CA (US)

(21) Appl. No.: 17/239,569

(22) Filed: Apr. 24, 2021

Related U.S. Application Data
(63) Continuation of application No. 14/932,904, filed on Nov. 4, 2015.
Application No. 14/932,904 is a continuation-in-part of application No. 14/698,665, filed on Apr. 28, 2015.
(60) Provisional application No. 61/985,406, filed on Apr. 28, 2014.

(51) Int. Cl. A61B 17/70; A61B 17/72; A61B 17/80

(52) U.S. Cl. CPC A61B 17/7016; A61B 17/7216; A61B 2017/00039; A61B 17/72; A61B 17/8004

(57) ABSTRACT

According to some embodiments, systems and methods are provided for non-invasively detecting the force generated by a non-invasively adjustable implantable medical device and/or a change in dimension of a non-invasively adjustable implantable medical device. Some of the systems include a non-invasively adjustable implant, which includes a driven magnet, and an external adjustment device, which includes one or more driving magnets and one or more Hall effect sensors. The Hall effect sensors of the external adjustment device are configured to detect changes in the magnetic field between the driven magnet of the non-invasively adjustable implant and the driving magnet(s) of the external adjustment device. Changes in the magnetic fields may be used to calculate the force generated by and/or a change in dimension of the non-invasively adjustable implantable medical device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

Scoliosis is a general term for the sideways (lateral) curving of the spine, usually in the thoracic or thoracolumbar region. Scoliosis is commonly broken up into different treatment groups, Adolescent Idiopathic Scoliosis, Early Onset Scoliosis and Adult Scoliosis.

Adolescent Idiopathic Scoliosis (AIS) typically affects children between ages 10 and 16, and becomes most severe during growth spurts that occur as the body is developing. One to two percent of children between ages 10 and 16 have some amount of scoliosis. Of every 1000 children, two to five develop curves that are serious enough to require treatment. The degree of scoliosis is typically described by the Cobb angle, which is determined, usually from x-ray images, by taking the most tilted vertebrae above and below the apex of the curved portion and measuring the angle between intersecting lines drawn perpendicular to the top of the top vertebra and the bottom of the bottom vertebra. The term idiopathic refers to the fact that the exact cause of this curvature is unknown. Some have speculated that scoliosis occurs during rapid growth phases when the ligamentum flavum of the spine is too tight and hinders symmetric growth of the spine. For example, as the anterior portion of the spine elongates faster than the posterior portion, the thoracic spine begins to straighten, until it curves laterally, often with an accompanying rotation. In more severe cases, this rotation actually creates a noticeable deformity, in which one shoulder is lower than the other. Currently, many school districts perform external visual assessment of spines, for example in all fifth grade students. For those students in whom an “S” shape or “C” shape is identified, instead of an “I” shape, a recommendation is given to have the spine examined by a physician, and commonly followed-up with periodic spinal x-rays.

Typically, patients with a Cobb angle of 20° or less are not treated, but are periodically monitored, often with subsequent x-rays. Patients with a Cobb angle of 400 or greater are usually recommended for fusion surgery. It should be noted that many patients do not receive this spinal assessment, for numerous reasons. Many school districts do not perform this assessment, and many children do not regularly visit a physician. So, the curve often progresses rapidly and severely. There is a large population of grown adults with untreated scoliosis, in extreme cases with a Cobb angle as high as or greater than 90°. Many of these adults, though, do not experience pain associated with this deformity, and live relatively normal lives, though oftentimes with restricted mobility and motion. In AIS, the ratio of females to males for curves under 10° is about one to one. However, at angles above 30°, females outnumber males by as much as eight to one. Fusion surgery can be performed on AIS patients or on adult scoliosis patients. In a typical posterior fusion surgery, an incision is made down the length of the back and Titanium or stainless steel straightening rods are placed along the curved portion of the spine. These rods are typically secured to the vertebral bodies, for example with hooks or bone screws (e.g., pedicle screws) in a manner that allows the spine to be straightened. Usually the intervertebral disks are removed and bone graft material is placed to create the fusion. If this is autologous material, the bone graft material is harvested from the patient's hip via a separate incision.

Alternatively, the fusion surgery may be performed anteriorly. Lateral and anterior incisions are made for access. Usually, one of the lungs is deflated in order to allow access to the spine. In a less-invasive version of the anterior procedure, instead of a single long incision, approximately five incisions, each about three to four cm long, are made in the intercostal spaces (between the ribs) on one side of the patient. In one version of this minimally invasive surgery, tethers and bone screws are placed and secured to the vertebra on the anterior convex portion of the curve. Clinical trials are being performed that use staples in place of the tether/screw combination. One advantage of this surgery, by comparison to the posterior approach is that the scars from the incisions are not as dramatic, though they are still located in a frequently visible area, (for example when a bathing suit is worn). Staples have experienced difficulty in clinical trials as they tend to pull out of the bone when a critical stress level is reached.

In some cases, after surgery, the patient will wear a protective brace for a few months as the fusing process occurs. Once the patient reaches spinal maturity, it is difficult to remove the rods and associated hardware in a subsequent surgery as the fusion of the vertebra usually incorporates the rods themselves. Standard practice is to leave the implants in for life. With either of these two surgical methods, after fusion, the patient's spine is straight, but depending on how many vertebrae were fused, there are often limitations in the degree of spinal flexibility, both in bending and twisting. As fused patients mature, the fused section can impart large stresses on the adjacent non-fused vertebra, and often other problems, including pain, can occur in these areas, sometimes necessitating further surgery. This tends to be in the lumbar portion of the spine that is prone to problems in aging patients. Many physicians are now interested in fusionless surgery for scoliosis, which may be able to eliminate some of the drawbacks of fusion.

One group of patients in which the spine is especially dynamic is the subset known as Early Onset Scoliosis (EOS), which typically occurs in children before the age of five, and more often in boys than in girls. While this is a comparatively uncommon condition, occurring in only about one or two out of 10,000 children, it can be severe, affecting the normal development of internal organs. Because of the fact that the spines of these children will still grow a large amount after treatment, non-fusion distraction devices known as growing rods and a device known as the VEPTR—Vertical Expandable Prosthetic Titanium Rib (“Titanium Rib”) have been developed. These devices are typically adjusted approximately every six months, to match the child's growth, until the child is at least eight years old, sometimes until they are 15 years old. Each adjustment requires a surgical incision to access the adjustable portion of the device. Because the patients may receive the device at an age as young as six months, this treatment may require a large number of surgeries thereby increasing the likelihood of infection for these patients.

The treatment methodology for AIS patients with a Cobb angle between 20° and 40° is controversial. Many physicians prescribe a brace (for example, the Boston Brace), that the patient must wear on their body and under their clothes 18 to 23 hours a day until they become skeletally mature, for example until age 16. Because these patients are all passing through their socially demanding adolescent years, it may be a serious prospect to be forced with the choice of: 1) either wearing a somewhat bulky brace that covers most of the upper body; 2) having fusion surgery that may leave large scars and also limit motion; 3) or doing nothing and running the risk of becoming disfigured and and/or disabled. It is commonly known that patients have hidden their braces, (in order to escape any related embarrassment) for example, in a bush outside of school. Patient compliance with braces has been so problematic that special braces have been designed to sense the body of the patient, and monitor the amount of time per day that the brace is worn. Even so, patients have been known to place objects into unworn braces of this type in order to fool the sensor. In addition with inconsistent patient compliance, many physicians believe that, even when used properly, braces are not effective in curing scoliosis. These physicians may agree that bracing can possibly slow, or even temporarily stop, curve (Cobb angle) progression, but they have noted that the scoliosis progresses rapidly, to a Cobb angle more severe than it was at the beginning of treatment, as soon as the treatment period ends and the brace is no longer worn. Some believe braces to be ineffective because they work only on a portion of the torso, rather than on the entire spine. A prospective, randomized 500 patient clinical trial known as BrAIST (Bracing in Adolescent Idiopathic Scoliosis Trial) is currently enrolling patients. 50% of the patients will be treated using a brace and 50% will simply be monitored. The Cobb angle data will be measured continually up until skeletal maturity, or until a Cobb angle of 50° is reached. Patients who reach a Cobb angle of 50° will likely undergo corrective surgery. Many physicians believe that the BrAIST trial will establish that braces are ineffective. If this is the case, uncertainty regarding how to treat AIS patients having a Cobb angle between 20° and 40° will only become more pronounced. It should be noted that the “20° to 40°” patient population is as much as ten times larger than the “40° and greater” patient population.

Distraction osteogenesis, also known as distraction callotasis and osteodistraction has been used successfully to lengthen long bones of the body. Typically, the bone, if not already fractured, is purposely fractured by means of a corticotomy, and the two segments of bone are gradually distracted apart, thereby allowing new bone to form in the gap. If the distraction rate is too high, there is a risk of nonunion, if the rate is too low, there is a risk that the two segments will completely fuse to each other before the distraction is complete. When the desired length of the bone is achieved using this process, the bone is allowed to consolidate. Distraction osteogenesis applications are mainly focused on the growth of the femur or tibia, but may also osteogenesis is mainly applied to growth of the femur or tibia, but may also include the humerus, the jaw bone (micrognathia), or other bones. Reasons for lengthening or growing bones are multifold and include, but are not limited to: post osteosarcoma bone cancer; cosmetic lengthening (both legs-femur and/or tibia) in short stature or dwarfism/achondroplasia; lengthening of one limb to match the other (congenital, post-trauma, post-skeletal disorder, prosthetic knee joint); and nonunions.

Distraction osteogenesis using external fixators has been done for many years, but the external fixator can be unwieldy for the patient. It can also be painful, and the patient is subject to the risk of pin track infections, joint stiffness, loss of appetite, depression, cartilage damage and other side effects. Having the external fixator in place also delays the beginning of rehabilitation.

In response to the shortcomings of external fixator distraction, intramedullary distraction nails have been surgically implanted which are contained entirely within the bone. Some are automatically lengthened via repeated rotation of the patient's limb, which can sometimes be painful to the patient and can often proceed in an uncontrolled fashion. This therefore makes it difficult to follow a strict daily or weekly lengthening regime that avoids nonunion (if too fast) or early consolidation (if too slow). Lower limb distraction may be about one mm per day. Other intramedullary nails have been developed which have an implanted motor that is remotely controlled by an antenna. These devices are designed to be lengthened in a controlled manner, but due to their complexity may not be manufacturable as an affordable commercial product. Others have proposed intramedullary distractors containing an implanted magnet that allows the distraction to be driven electromagnetically by an external stator. Because of the complexity and size of the external stator, this technology has not been reduced to a simple, cost-effective device that can be taken home, to allow patients to do daily lengthenings. Non-invasively (magnetically) adjustable implantable distraction devices have been developed and use clinically in both scoliosis patients and in limb lengthening patients.

Knee osteoarthritis is a degenerative disease of the knee joint that affects a large number of patients, particularly over the age of 40. The prevalence of this disease has increased significantly over the last several decades, attributed partially, but not completely, to the rising age of the population and the increase in obesity. The increase may also be due partially to an increasing number of highly active people within the population. Knee osteoarthritis is caused mainly by long term stresses on the knee that degrade the cartilage covering the articulating surfaces of the bones in the knee joint. Oftentimes, the problem becomes worse after a particular trauma event, but it can also be a hereditary process. Symptoms may include pain, stiffness, reduced range of motion, swelling, deformity, muscle weakness, and several others. Osteoarthritis may include one or more of the three compartments of the knee: the medial compartment of the tibiofemoral joint, the lateral compartment of the tibiofemoral joint, and the patellofemoral joint. In severe cases, partial or total replacement of the knee is performed in order to replace the degraded/diseased portions with new weight bearing surfaces for the knee. These implants are typically made from implant grade plastics, metals, or ceramics. Replacement operations may involve significant post-operative pain and require substantial physical therapy. The recovery period may last weeks or months. Several potential complications of this surgery exist, including deep venous thrombosis, loss of motion, infection and bone fracture. After recovery, surgical patients who have received uni-compartmental or total knee replacement must significantly reduce their activity, removing running and high energy sports completely from their lifestyle.

For these reasons, surgeons may attempt to intervene early in order to delay or even preclude knee replacement surgery. Osteotomy surgeries may be performed on the femur or tibia to change the angle between the femur and tibia, thereby adjusting the stresses on the different portions of the knee joint. In closed wedge and closing wedge osteotomy, an angled wedge of bone is removed and the remaining surfaces are fused together to create a new, improved bone angle. In open wedge osteotomy, a cut is made in the bone and the edges of the cut are opened, creating a new angle. Bone graft is often used to fill in the new opened wedge-shaped space, and, often, a plate is attached to the bone with bone screws. Obtaining the correct angle during either of these types of osteotomy is almost always difficult, and even if the result is close to what was desired, there can be a subsequent loss of the correction angle. Other complications experienced with this technique may include nonunion and material failure.

In addition to the many different types of implantable distraction devices that are configured to be non-invasively adjusted, implantable non-invasively adjustable non-distraction devices have also been envisioned, for example, adjustable restriction devices for gastrointestinal disorders such as GERD, obesity, or sphincter laxity (such as in fecal incontinence), or other disorders such as sphincter laxity in urinary incontinence. These devices too may incorporate magnets to enable the non-invasive adjustment.

### SUMMARY

In some embodiments, a remote control for adjusting a medical implant includes a driver, at least one sensor, and an output. The driver is configured to transmit a wireless drive signal to adjust an implanted medical implant. Adjustment of the medical implant includes one or more of generating a force with the medical implant and changing a dimension of the medical implant. The at least one sensor is configured to sense a response of the implant to the drive signal. The output is configured to report one or more of a force generated by the medical implant and a change in dimension of the medical implant, in response to the drive signal. In some embodiments, the output is a visual output (e.g., a display), an audio output (e.g., a speaker, alarm), a USB output, a Bluetooth output, a solid state memory output (e.g., any removable or readable solid state memory), etc.

In some embodiments, a medical implant for wireless adjustment of a dimension within a body includes a first portion that is configured for coupling to a first location in the body, a second portion that is configured for coupling to a second location in the body, and a magnetic drive that is configured to adjust a relative distance between the first portion and the second portion. The magnetic drive includes at least one driven magnet and is configured to revolve about an axis in response to a magnetic field imposed by a rotatable driver magnet outside of the body. The implant is configured to transmit a signal indicative of the responsiveness of the driven magnet to movement of the driver magnet, wherein a change in the responsiveness is indicative of a change in a force applied by the body to the first and second connectors.

### BRIEF DESCRIPTION OF THE DRAWINGS

FIG. 1 illustrates one embodiment of an external adjustment device.

FIG. 2 illustrates a detailed view of the display and control panel of the external adjustment device of FIG. 1.

FIG. 3 illustrates the lower or underside surfaces of the external adjustment device of FIG. 1.

FIG. 4 illustrates a sectional view of the external adjustment device of FIG. 3 taken along line 4-4 of FIG. 3.

FIG. 5 illustrates a sectional view of the external adjustment device of FIG. 3 taken along line 5-5 of FIG. 3.

FIG. 6 illustrates an orientation of magnets of one embodiment of an external adjustment device in relation to a magnet of a distraction device.

FIG. 7 illustrates various sensors on a circuit board of one embodiment of the external adjustment device.

FIG. 8 illustrates various Hall effect sensors on a circuit board of one embodiment of the external adjustment device.

FIG. 9A illustrates a particular configuration of Hall effect sensors relating to the magnets of one embodiment of an external adjustment device.

FIG. 9B illustrates output voltage of the Hall effect sensors of FIG. 9A.

FIG. 9C illustrates the Hall effect sensors of FIG. 9A, with the magnets in a nonsynchronous condition.

FIG. 9D illustrates the output voltage of the Hall effect sensors of FIG. 9C.

FIG. 10A illustrates a configuration of Hall effect sensors relating to the magnets of one embodiment.

FIG. 10B illustrates the output voltage of the Hall effect sensors of FIG. 10A.

FIG. 11 illustrates a magnetic flux density plot of external magnets of one embodiment of an external adjustment device and the internal permanent magnet.

FIG. 12A illustrates a section view of external magnets of one embodiment of an external adjustment device and the internal permanent magnet during positioning of the external adjustment device.

FIG. 12B illustrates a side view of external magnets of one embodiment of an external adjustment device and the internal permanent magnet during positioning of the external adjustment device.

FIG. 12C illustrates a top view of external magnets of one embodiment of an external adjustment device and the internal permanent magnet during positioning of the external adjustment device.

FIG. 13A illustrates a zero torque condition between external magnets of one embodiment of an external adjustment device and the internal permanent magnet.

FIG. 13B illustrates magnetic coupling between external magnets of one embodiment an external adjustment device and the internal permanent magnet.

FIG. 13C illustrates continued rotation with increasing coupling torque between external magnets of one embodiment of an external adjustment device and the internal permanent magnet.

FIG. 13D illustrates slippage between external magnets of one embodiment of an external adjustment device and the internal permanent magnet.

FIG. 14 is an internal view of one embodiment of an external adjustment device having an array of magnetic sensors.

FIG. 15 is a circuit board containing magnetic sensors.

FIG. 16 is a front view of one embodiment of an external adjustment device having an array of magnetic sensors.

FIG. 17 is a front view of an arrangement of magnetic sensors in relation to external magnets of one embodiment of an external adjustment device and an internal permanent magnet.

FIG. 18 is a sectional view of the arrangement of magnetic sensors of FIG. 17 taken along line 18.

FIG. 19 is a system diagram of one embodiment of an external adjustment device of a system for adjusting an adjustable implant.

FIG. 20 is a block diagram of the logic sequence for one embodiment of an external adjustment device of a system for adjusting an adjustable implant.

FIG. 21 is a user interface for one embodiment of an external adjustment device of a system for adjusting an adjustable implant.

FIG. 22 is a graph of voltage over a series of gap distances.

FIG. 23 is a graph of maximum possible distraction force over a series of gap distances.

FIG. 24 is a graph of actual force for several voltage differentials.

FIG. 25 is a graph of differential voltages of pairs of magnetic sensors.

FIG. 26 illustrates an embodiment of an adjustable implant for adjusting length of or force on a spine.

FIG. 27 is an embodiment of an adjustable implant for adjusting the distance or force between sections of bone.

FIG. 28 is an embodiment of an adjustable implant for adjusting a rotational angle or torque between sections of bone.

FIG. 29 is an embodiment of an adjustable implant for adjusting an angle or force between sections of bone.

FIG. 30 is an embodiment of an adjustable implant for adjusting an angle or force between sections of bone.

FIG. 31 is an embodiment of an adjustable implant for adjusting a location or force (tension) on body tissue.

FIG. 32 is an embodiment of an adjustable implant for adjusting restriction on a duct of the body.

FIG. 33 is a front view of an arrangement of magnetic sensors in relation to one or more external electromagnets of one embodiment of an external adjustment device and an internal permanent magnet.

FIG. 34 is a partial sectional view of an array of magnetic sensors in relation to external magnets of one embodiment of an external adjustment device and an internal permanent magnet.

FIG. 35 is a front view of an arrangement of magnetic sensors in an embodiment of an external adjustment device.

FIG. 36 is a graph of actual force against voltage differential for two different gap distances.

FIG. 37A is an embodiment of an adjustable implant incorporating a magnet into the lead screw.

FIG. 37B is an embodiment of an adjustable implant incorporating a magnet into the distraction rod.

FIG. 38 is an array of magnetic sensors for use with an embodiment of an adjustable implant.

FIG. 39 shows multiple arrays of magnetic sensors for use with embodiments of an adjustable implant.

FIG. 40 is a front view of a magnetic sensor in an embodiment of an external adjustment device.

FIG. 41 is an arrangement of magnetic sensors in an embodiment of an external adjustment device.

FIGS. 42A & 42B show a wire coil for use with an embodiment of an external adjustment device.

FIG. 43 shows an embodiment of an external adjustment device having two wire coils being used on two adjustable implants implanted within a patient.

FIG. 44 illustrates a graph of a signal generated based on magnetic flux through a wire coil of an embodiment of the external adjustment device.

FIG. 45 illustrates graphs of signals generated based on magnetic flux through two wire coils of an embodiment of the external adjustment device.

### DETAILED DESCRIPTION

A detailed description of the broad concepts in this disclosure is provided in the following paragraphs. This description is directed to various example embodiments that are intended to be non-limiting, and the description is provided to facilitate understanding of the disclosure.

An external adjustment device is configured to adjust a medical implant by using a pair of rotating external magnets to rotate an internal magnet within the medical implant, causing the implant to be distracted (e.g., extend in length) or retracted (e.g., decrease in length). For example, the medical implant can be implanted next to the spine, and the external adjustment device can be used to non-invasively distract or retract the implant in order to affect the curvature of the spine. Alternatively, the medical implant could be implanted within a medullary canal of a long bone and used to affect the length or rotational orientation of the long bone, for example, the relative distance between two separate portions of the long bone or the relative degree of orientation between two separate portions of the long bone.

The external adjustment device may be configured to measure, either direct or indirectly, the distraction length of a medical implant. Measuring the distraction length may be accomplished by using magnetic sensors, such as Hall effect sensors or wire coils, contained within the external adjustment device. Such magnetic sensors may be able to detect the magnetic field of the internal magnet within the medical implant.

In a differential mode, this process involves positioning Hall effect sensors on the top and the bottom of the external adjustment device (e.g., near the top of the rotating magnets of the external adjustment device and near the bottom of the rotating magnets of the external adjustment device). A sensor pair may include a sensor at the top of the device and a sensor at the bottom of the device. Both sensors in a sensor pair may pick up on, register, or detect the magnetic field(s) associated with the pair of rotating magnets within the external adjustment device. However, the top sensor is further away from the medical implant and therefore picks up/detects primarily the magnetic field(s) of the pair of rotating magnets in the external adjustment device. By contrast, the bottom sensor is comparatively closer to the medical implant and therefore picks up/detects the magnetic field(s) of any internal magnet within any medical implant(s). Subtracting the signal value of the top sensor from the signal value of the bottom sensor in a sensor pair may be considered approximately equivalent to subtracting out the magnetic fields of the rotating magnets, leaving a signal value reflecting the magnetic field of the medical implant. This differential sensor configuration allows the external adjustment device to remotely measure certain values associated with the medical implant, and it may allow a user to draw certain inferences/conclusions about the implant without direct visual confirmation of the implant. Thus, the implant can advantageously be monitored in a non-invasive manner. For example, the differential signal may be used to determine whether the external adjustment device is close enough to the medical implant that they are magnetically “coupled”.

When the external adjustment device is magnetically coupled with the medical implant, rotation of the magnets within the external adjustment device causes rotation of the internal magnet of the implant, thereby causing the implant to distract or retract (depending on the direction of magnet rotation), or to increase in length or decrease in length. The field strength of a magnetic dipole drops off as a function of approximately r. So, the external adjustment device should be kept at a reasonably close distance to the medical implant to maintain a strong magnetic coupling (e.g., the field strength sufficiently high) such that the external adjustment device may rotate the internal magnet of the implant. To achieve this, the external adjustment device may be configured only to operate when it is close enough to the medical implant, with the software of the external adjustment device configurable to set a threshold value (and thus measurement sensitivity) for which the device is considered to be adequately coupled.

Differential signal (e.g., differential voltage) can also be used to estimate how much distraction or retraction force an implant is generating and/or delivering. The distance between the bottom of the external magnets to the top of the internal magnet within the implant (i.e., the “gap distance”) may be used to determine the amount of force an implant is generating. The gap distance may be estimated in a variety of ways, such as by medical imaging scans. For a given gap distance, there exists a relationship between the differential signal and the force generated. Thus, data may be collected for the relationship between differential signal and force for various different gap distances and a predictive model built for gap distance. The differential signal may also be used to determine whether the implant is stalling or slipping. Stalling or slipping occurs when the medical implant is experiencing a resistance force (e.g., from the body of the patient) greater than the magnetic coupling that cannot be overcome as the implant is being distracted.

Non-invasive measurement of the distraction length of the medical implants can be achieved through indirect measurement (e.g., by counting the rotations of the magnets within the external adjustment device). There may exist a relationship between the rotations of the magnets of the external adjustment device and the change in distraction length of an implant that can be measured and determined in advance. For example, the magnets of the external adjustment device rotate the internal magnet of the implant at a fixed ratio, which in turn rotates a screw in the implant at a fixed ratio, which then distracts or retracts the implant at a fixed ratio. In other words, by counting the revolutions of the magnets of the external adjustment device, it is possible to indirectly estimate the implant's distraction or retraction. This inference requires the assumptions that the external adjustment device is coupled (and rotating the internal magnet of the implant) and that the implant has not stalled. The differential signal from the sensor configuration may allow these assumptions to be confirmed, as described above. In some embodiments, only the rotations of the magnets for which there was coupling and no stalling may be considered in calculating the distraction length of the implant.

Further complexity in this disclosure is associated with the addition of medical implants and more direct methods of determining distraction length.

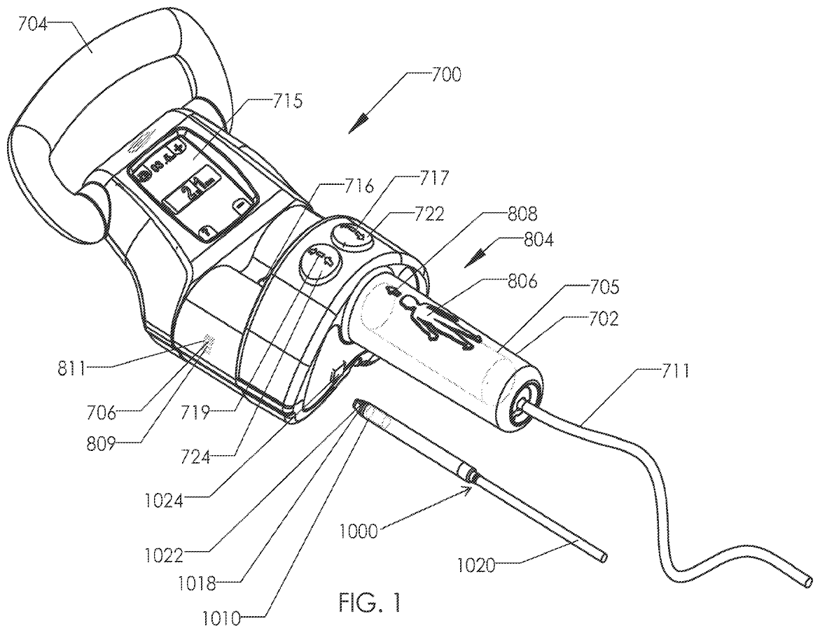

FIGS. 1-3 illustrate an external adjustment device 700 that is configured for adjusting an adjustable implant, such as a force-applying device, more specifically represented by (though not limited to) a distraction device. 1000 The distraction device 1000 may include any number of distraction, or generally, adjustable force-applying devices such as those described in U.S. Pat. Nos. 7,862,502, 7,955,357, 8,197,490, 8,449,543, and 8,852,187, the disclosures of which are hereby incorporated by reference in their entirety, and/or U.S. patent application Ser. Nos. 12/121,355, 12/411,107, 12/250,442, 12/761,141, 13/198,571, 13/655,246, 14/065,342, 13/791,430, 14/355,202, 14/447,391, and 14/511,084, the disclosures of which are hereby incorporated by reference in their entirety. The distraction device 1000 generally includes a rotationally mounted, internal permanent magnet 1010 that rotates in response to a magnetic field applied by the external adjustment device 700. Rotation of the magnet 1010 in one direction causes distraction of the device 1000 while rotation of the magnet 1010 in the opposite direction causes retraction of the device 1000. Retraction of the device 1000 may generate compressive force while distraction of the device 1000 may generate tensile forces. The external adjustment device 700 may be powered by a rechargeable battery or by a power cord 711. The external adjustment device 700 includes a first handle 702 and a second handle 704. The second handle 704 is in a looped shape, and can be used to carry the external adjustment device 700 and/or steady the external adjustment device 700 during use. The first handle 702 extends linearly from a first end of the external adjustment device 700 while the second handle 704 is located at a second end of the external adjustment device 700 and extends substantially off axis or is angled with respect to the first handle 702. In one embodiment, the second handle 704 may be oriented substantially perpendicular relative to the first handle 702, although other arrangements are possible.

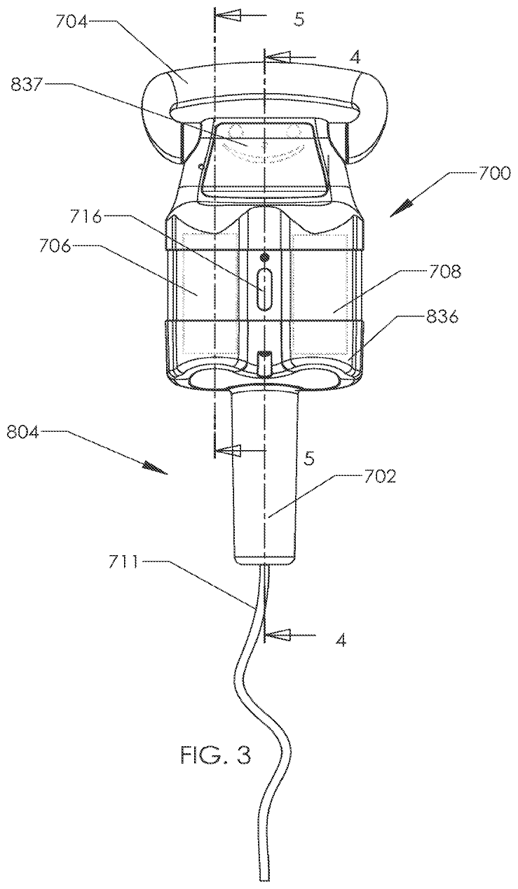

The first handle 702 contains a motor 705 that drives a first external magnet 706 and a second external magnet 708, best seen in FIG. 3, via gearing, belts or the like. On the first handle 702 is an optional orientation image 804 comprising a body outline 806 and an optional orientation arrow 808 that shows the correct direction to place the external adjustment device 700 on the patient's body, so that the distraction device is operated in the correct direction. While holding the first handle 702, the operator presses with his thumb the distraction button 722, which has a distraction symbol 717 and is a first color (e.g., green). This distracts the distraction device 1000. If the distraction device 1000 is over-distracted and it is desired to retract, or to lessen the distraction of the device 1000, the operator presses with his thumb the retraction button 724 which has a retraction symbol 719.

Distraction turns the magnets 706, 708 in one direction while retraction turns the magnets 706, 708 in the opposite direction. Magnets 706, 708 have stripes 809 that can be seen in window 811. This allows easy identification of whether the magnets 706, 708 are stationary or turning, and in which direction they are turning, as well as quick trouble shooting by the operator of the device. The operator can determine the point on the patient where the magnet of the distraction device 1000 is implanted, and then place the external adjustment device 700 in a correct location with respect to the distraction device 1000, by marking the corresponding portion of the skin of the patient, and then viewing this spot through an alignment window 716 of the external adjustment device 700.

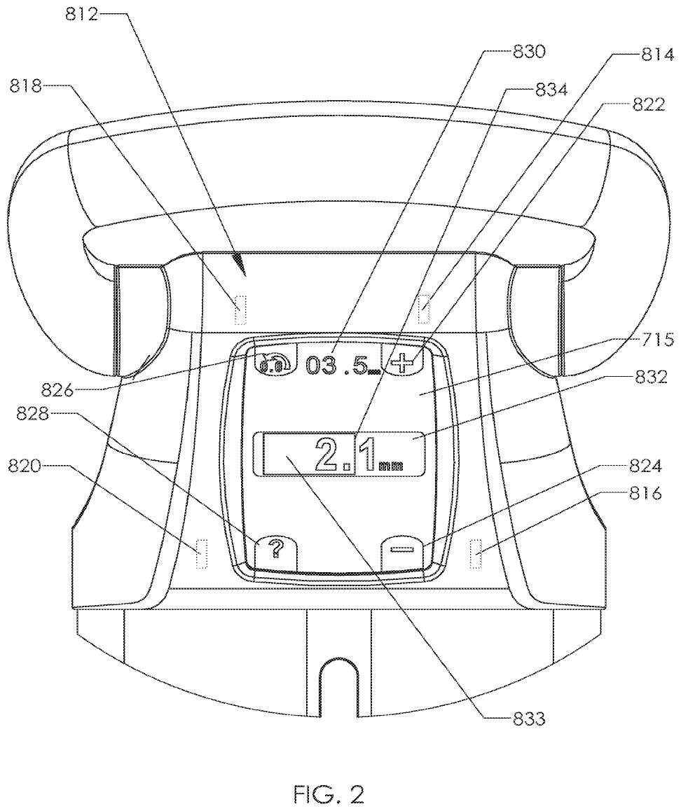

FIG. 2 illustrates a control panel 812 that includes several buttons 814, 816, 818, 820 and a display 715. The buttons 814, 816, 818, 820 are soft keys, and able to be programmed for an array of different functions. In some embodiments, the buttons 814, 816, 818, 820 have corresponding legends which appear in the display. To set the length of distraction to be performed on the distraction device 1000, the target distraction length 830 is adjusted using an increase button 814 and/or a decrease button 816. The legend with a green plus sign graphic 822 corresponds to the increase button 814 and the legend with a red negative sign graphic 824 corresponds to the decrease button 816. It should be understood that mention herein to a specific color used for a particular feature should be viewed as illustrative. Colors other than those specifically recited herein may be used in connection with the inventive concepts described herein. Each time the increase button 814 is depressed, it causes the target distraction length 830 to increase by 0.1 mm. In the same way each time the decrease button 816 is depressed, it causes the target distraction length 830 to decrease by 0.1 mm. Decrements/increments other than 0.1 mm could also be used. When the desired target distraction length 830 is displayed, and the external adjustment device 700 is placed on the patient, the operator holds down the distraction button 722, and the External Distraction Device 700 turns magnets 706, 708 until the target distraction length 830 is achieved (at which point the external adjustment device 700 stops). During the distraction process, the actual distraction length 832 is displayed, starting at 0.0 mm and increasing/decreasing until the target distraction length 830 is achieved. As the actual distraction length 832 increases/decreases, a distraction progress graphic 834 is displayed. For example a light colored box 833 that fills with a dark color from the left to the right. In FIG. 2, the target distraction length 830 is 3.5 mm, 2.1 mm of distraction has occurred, and 60% of the box 833 of the distraction progress graphic 834 is displayed. A reset button 818 corresponding to a reset graphic 826 can be pressed to reset one or both of the numbers back to zero. An additional button 820 can be assigned for other functions (e.g., help, data, etc.). This button can have its own corresponding graphic 828 (shown in FIG. 2 as “?”). Alternatively, a touch screen can be used, for example capacitive or resistive touch keys. In this embodiment, the graphics/legends 822, 824, 826, 828 may also be touch keys, replacing or augmenting the buttons 814, 816, 818, 820. In one particular embodiment, touch keys at 822, 824, 826, 828 perform the functions of buttons 814, 816, 818, 820 respectively, and the buttons 814, 816, 818, 820 are eliminated. In some embodiments, outputs other than a display may be used, including, for example, an audio output, a USB output, a Bluetooth output, or any other data output that can effectively report data resulting from use of the external adjustment device 700 to a user.

Handles 702, 704 can be held in several ways. For example the first handle 702 can be held with palm facing up while trying to find the location on the patient of the implanted magnet of the distraction device 1000. The fingers are wrapped around the handle 702 and the fingertips or mid-points of the four fingers press up slightly on the handle 702, balancing it somewhat. This allows a very sensitive feel that allows the magnetic field between the magnet in the distraction device 1000 and the magnets 706, 708 of the external adjustment device 700 to be more apparent. During the distraction, the first handle 702 may be held with the palm facing down, allowing the operator to push the device 700 down firmly onto the patient, to minimize the distance between the magnets 706, 708 of the external adjustment device 700 and the magnet 1010 of the distraction device 1000, and thus maximizing the torque coupling. This is especially appropriate if the patient is large or overweight. The second handle 704 may be held with the palm up or the palm down during the magnet sensing operation and the distraction operation, depending on the preference of the operator.

FIG. 3 illustrates the underside, or lower surface, of the external adjustment device 700. At the bottom of the external adjustment device 700, the contact surface 836 may be made of material of a soft durometer, such as an elastomeric material, for example PEBAX® (Arkema, Inc., Torrance, Calif., USA) or Polyurethane. This allows for anti-shock to protect the device 700 if it is dropped. Also, if placing the device on patient's bare skin, materials of this nature do not pull heat away from patient as quickly as some other materials; hence, they “don't feel as cold” as hard plastic or metal. The handles 702, 704 may also have similar material covering them, in order to serve as non-slip grips.

FIG. 3 also illustrates child-friendly graphics 837, including the option of a smiley face. Alternatively this could be an animal face, such as a teddy bear, a horsey, or a bunny rabbit. A set of multiple faces can be removable and interchangeable to match the likes of various young patients. In addition, the location of the faces on the underside of the device allows the operator to show the faces to a younger child, but keep it hidden from an older child, who may not be so amused. Alternatively, sock puppets or decorative covers featuring human, animal, or other characters may be produced so that the device may be thinly covered with them, without affecting the operation of the device, but additionally, the puppets or covers may be given to the young patient after a distraction procedure is performed. It is expected that this can help keep a young child more interested in returning to future procedures.

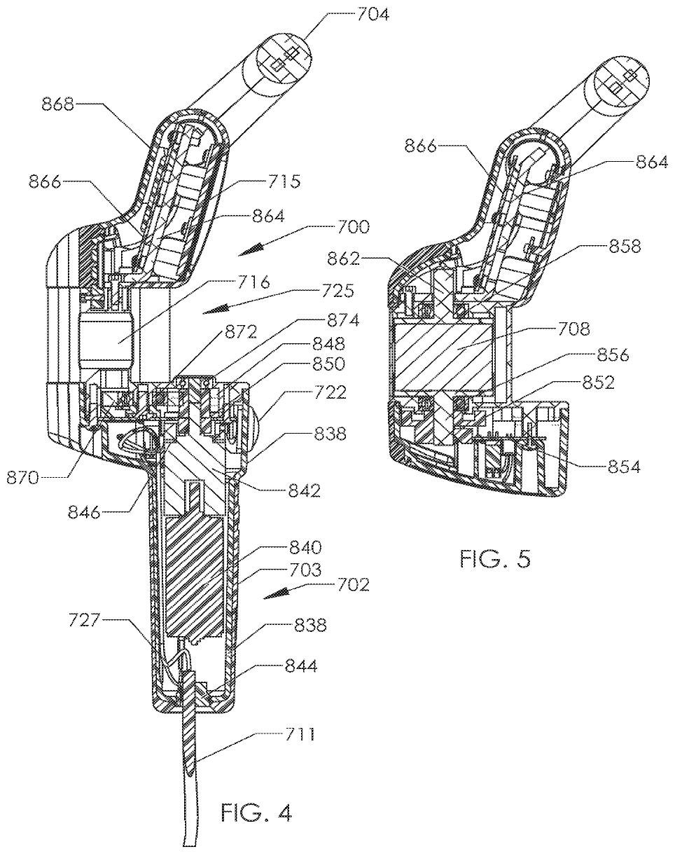

FIGS. 4 and 5 are sectional views of the external adjustment device 700 shown in FIG. 3, which illustrate the internal components of the external adjustment device 700 taken along various centerlines. FIG. 4 is a sectional view of the external adjustment device 700 taken along the line 4-4 of FIG. 3. FIG. 5 is a sectional view of the external adjustment device 700 taken along the line 5-5 of FIG. 3. The external adjustment device 700 comprises a first housing 868, a second housing 838 and a central magnet section 725. First handle 702 and second handle 704 include grip 703 (shown on first handle 702). Grip 703 may be made of an elastomeric material and may have a soft feel when gripped by the hand. The material may also have a tacky feel, in order to aid firm gripping. Power is supplied via power cord 711, which is held to second housing 838 with a strain relief 844. Wires 727 connect various electronic components including motor 840, which rotates magnets 706, 708 via gear box 842, output gear 848, and center gear 870 respectively. Center gear 870 rotates two magnet gears 852, one on each magnet 706, 708 (one such gear 852 is illustrated in FIG. 5). Output gear 848 is attached to motor output via coupling 850, and both motor 840 and output gear 848 are secured to second housing 838 via mount 846. Magnets 706, 708 are held within magnet cups 862. Magnets and gears are attached to bearings 872, 874, 856, 858, which aid in low friction rotation. Motor 840 is controlled by motor printed circuit board (PCB) 854, while the display is controlled by display PCB 866, which is attached to frame 864.

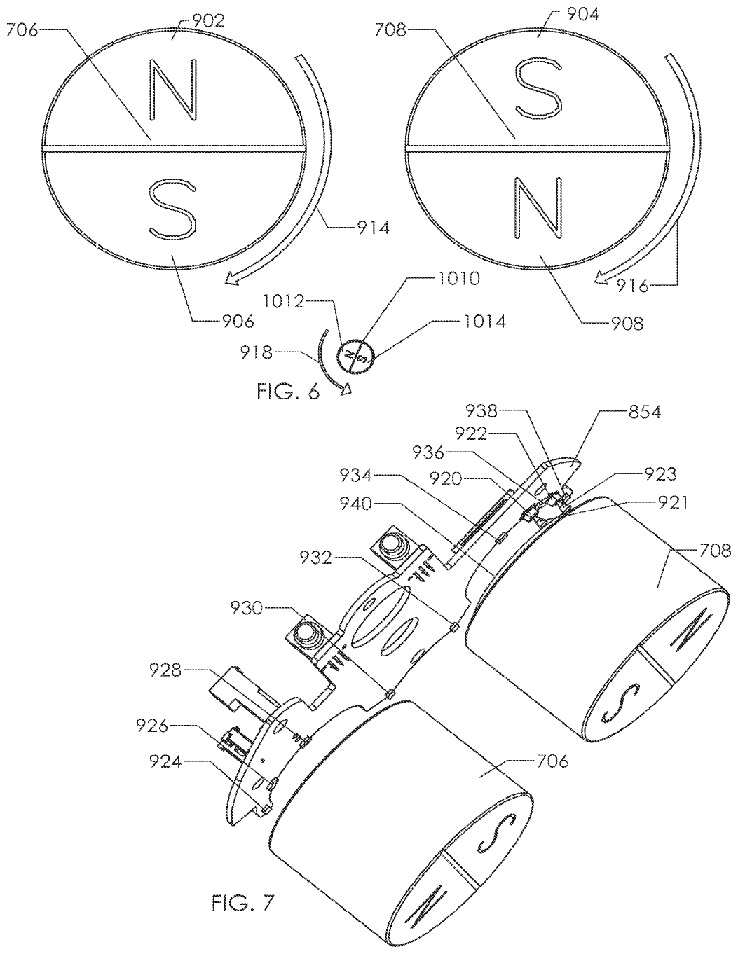

FIG. 6 illustrates the orientation of poles of the first and second external magnets 706, 708 and the implanted magnet 1010 of the distraction device 1000 during a distraction procedure. For the sake of description, the orientations will be described in relation to the numbers on a clock. First external magnet 706 is turned (by gearing, belts, etc.) synchronously with second external magnet 708 so that north pole 902 of first external magnet 706 is pointing in the twelve o'clock position when the south pole 904 of the second external magnet 708 is pointing in the twelve o'clock position. At this orientation, therefore, the south pole 906 of the first external magnet 706 is pointing is pointing in the six o'clock position while the north pole 908 of the second external magnet 708 is pointing in the six o'clock position. Both first external magnet 706 and second external magnet 708 are turned in a first direction as illustrated by respective arrows 914, 916. The rotating magnetic fields apply a torque on the implanted magnet 1010, causing it to rotate in a second direction as illustrated by arrow 918. Exemplary orientation of the north pole 1012 and south pole 1014 of the implanted magnet 1010 during torque delivery are shown in FIG. 6. When the first and second external magnets 706, 708 are turned in the opposite direction from that shown, the implanted magnet 1010 will be turned in the opposite direction from that shown. The orientation of the first external magnet 706 and the second external magnet 708 in relation to each other serves to optimize the torque delivery to the implanted magnet 1010. During operation of the external adjustment device 700, it is often difficult to confirm that the two external magnets 706, 708 are being synchronously driven as desired.

Turning to FIGS. 7 and 8, in order to ensure that the external adjustment device 700 is working properly, the motor printed circuit board 854 comprises one or more encoder systems, for example photointerrupters 920, 922 and/or Hall effect sensors 924, 926, 928, 930, 932, 934, 936, 938. Photointerrupters 920, 922 each comprise an emitter and a detector. A radially striped ring 940 may be attached to one or both of the external magnets 706, 708 allowing the photointerrupters to optically encode angular motion. Light 921, 923 is schematically illustrated between the radially striped ring 940 and photointerrupters 920, 922.

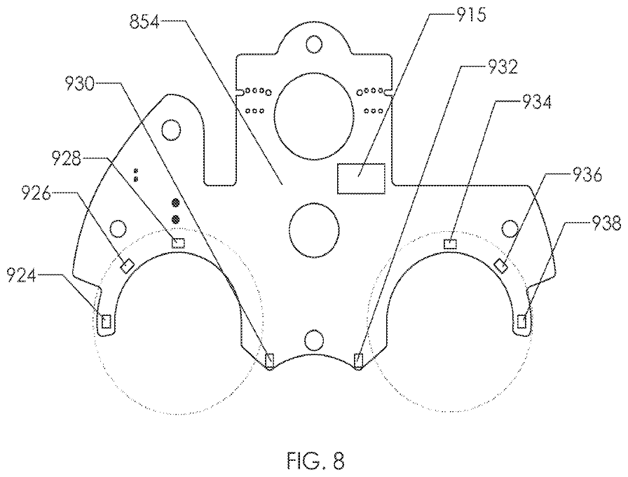

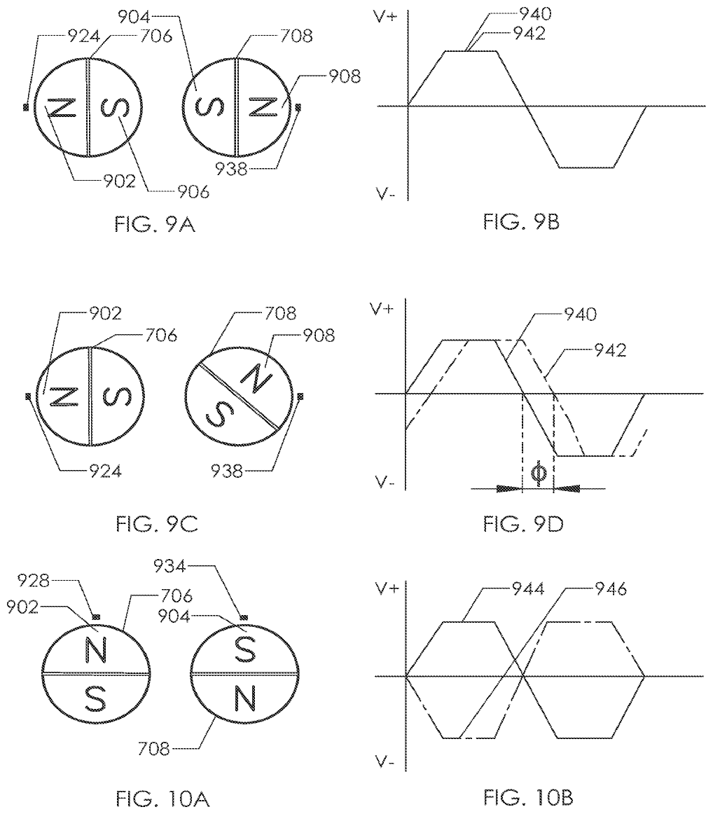

Independently, Hall effect sensors 924, 926, 928, 930, 932, 934, 936, 938 may be used as non-optical encoders to track rotation of one or both of the external magnets 706, 708. While eight (8) such Hall effect sensors are illustrated in FIG. 7, it should be understood that fewer or more such sensors may be employed. The Hall effect sensors are connected to the motor printed circuit board 854 at locations that allow the Hall effect sensors to sense the magnetic field changes as the external magnets 706, 708 rotate. Each Hall effect sensor 924, 926, 928, 930, 932, 934, 936, 938 outputs a voltage that corresponds to increases or decreases in the magnetic field strength. FIG. 9A indicates one basic arrangement of Hall effect sensors relative to sensors 924, 938. A first Hall effect sensor 924 is located at nine o'clock in relation to first external magnet 706. A second Hall effect sensor 938 is located at three o'clock in relation to second external magnet 708. As the magnets 706, 708 rotate in synchronous motion, the first voltage output 940 of first Hall effect sensor 924 and second voltage output 942 of second Hall effect sensor 938 have the same pattern, as seen in FIG. 9B, which graphs voltage for a full rotation cycle of the external magnets 706, 708. The graph indicates a sinusoidal variance of the output voltage, but the clipped peaks are due to saturation of the signal. Even if Hall effect sensors used in the design cause this effect, there is still enough signal to compare the first voltage output 940 and the second voltage output 942 over time. If either of the two Hall effect sensors 924, 938 does not output a sinusoidal signal during the operation or the external adjustment device 700, this demonstrates that the corresponding external magnet has stopped rotating. FIG. 9C illustrates a condition in which both the external magnets 706, 708 are rotating at the same approximate angular speed, but the north poles 902, 908 are not correctly synchronized. Because of this, the first voltage output 940 and second voltage output 942 are out-of-phase, and exhibit a phase shift (ϕ). These signals are processed by a processor 915 (shown in FIG. 8) and an error warning is displayed on the display 715 of the external adjustment device 700 so that the device may be resynchronized.

If independent stepper motors are used, the resynchronization process may simply be one of reprogramming, but if the two external magnets 706, 708 are coupled together, by gearing or a belt for example, a mechanical rework may be required. An alternative to the Hall effect sensor configuration of FIG. 9A is illustrated in FIG. 10A. In this embodiment, Hall effect sensor 928 is located at twelve o'clock in relation to external magnet 706 and Hall effect sensor 934 is located at twelve o'clock in relation to external magnet 708. With this configuration, the north pole 902 of external magnet 706 should be pointing towards Hall effect sensor 928 when the south pole 904 of external magnet 708 is pointing towards Hall effect sensor 934. With this arrangement, Hall effect sensor 928 outputs output voltage 944 and Hall effect sensor 934 outputs output voltage 946 (FIG. 10B). Output voltage 944 is, by design, out of phase with output voltage 946. An advantage of the Hall effect sensor configuration of FIG. 9A is that the each sensor has a larger distance between it and the opposite magnet (e.g., Hall effect sensor 924 in comparison to external magnet 708) so that there is less possibility of interference. An advantage to the Hall effect sensor configuration of FIG. 10A is that it may be possible to make a more compact external adjustment device 700 (less width). The out-of-phase pattern of FIG. 10B can also be analyzed to confirm magnet synchronicity.

Returning to FIGS. 7 and 8, additional Hall effect sensors 926, 930, 932, 936 are shown. These additional sensors allow additional precision to the rotation angle feedback of the external magnets 706, 708 of the external adjustment device 700. Again, the particular number and orientation of Hall effect sensors may vary. In place of the Hall effect sensors, magnetoresistive encoders may also be used.

In still another embodiment, additional information may be processed by processor 915 and may be displayed on display 715. For example, distractions using the external adjustment device 700 may be performed in a doctor's office by medical personnel, or by patients or members of patient's family in the home. In either case, it may be desirable to store information from each distraction session to be accessed later. For example, the date and time of each distraction, the amount of distraction attempted, and the amount of distraction obtained. This information may be stored in the processor 915 or in one or more memory modules (not shown) associated with the processor 915. In addition, the physician may be able to input distraction length limits, for example the maximum amount that can be distracted in each session, the maximum amount that can be distracted per day, the maximum amount that can be distracted per week, etc. The physician may input these limits by using a secure entry using the keys or buttons of the device, which the patient will not be able to access.

Returning to FIG. 1, in some patients, it may be desired to place a first end 1018 of the distraction device 1000 towards the head of the patient, and second end 1020 of the distraction device 1000 towards the feet of the patient. This orientation of the distraction device 1000 may be termed antegrade. In other patients, it may be desired to orient the distraction device 1000 with the second end 1020 of the distraction device 1000 towards the head of the patient, and the first end 1018 of the distraction device 1000 towards the feet of the patient. This orientation of the distraction device 1000 may be termed retrograde. In a distraction device 1000 in which the magnet 1010 rotates in order to turn a screw within a nut, the orientation of the distraction device 1000 being either antegrade or retrograde in patient could mean that the external adjustment device 700 would have to be placed in accordance with the orientation image 804 when the distraction device 1000 is placed antegrade, but placed the opposite of the orientation image 804 when the distraction device 1000 is placed retrograde. Software may be programmed so that the processor 915 recognizes whether the distraction device 1000 has been implanted antegrade or retrograde, and then turns the magnets 706, 708 in the appropriate direction when the distraction button 722 is placed.

For example, the motor 705 could be commanded to rotate the magnets 706, 708 in a first direction when distracting an antegrade placed distraction device 1000, and in a second, opposite direction when distracting a retrograde placed distraction device 1000. The physician may, for example, be prompted by the display 715 to input using the control panel 812 whether the distraction device 1000 was placed antegrade or retrograde. The patient may then continue to use the same external adjustment device 700 to assure that the motor 705 turns the magnets 706, 708 in the proper directions for both distraction and refraction. Alternatively, the distraction device may incorporate an RFID chip 1022 (shown in FIG. 1), which can be read and written to by an antenna 1024 on the external adjustment device 700. The position of the distraction device 1000 in the patient (antegrade or retrograde) can be written to the RFID chip 1022, and can thus be read by the antenna 1024 of any external adjustment device 700, allowing the patient to receive correct distractions and/or retractions, regardless of which external adjustment device 700 is used.

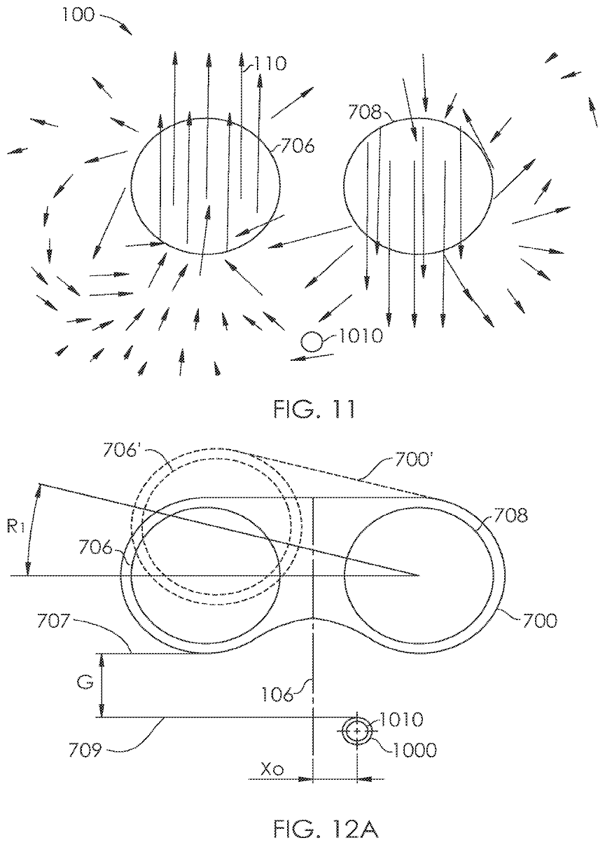

FIG. 11 is a magnetic flux density plot 100 of the magnetic field characteristics in the region surrounding the two external magnets 706, 708 of the external adjustment device 700, and the internal permanent magnet 1010 of the distraction device 1000. For the purposes of this disclosure, any type of adjustable force-applying (or torque-applying) implant incorporating a rotatable magnet is contemplated as an alternative. In the flux density plot 100, a series of flux lines 110 are drawn as vectors, having orientation and magnitude, the magnitude represented by the length of the arrows. As the external magnets 706, 708 magnetically couple with the internal permanent magnet 1010 and are turned by the motor 840 (FIG. 4) causing the internal permanent magnet 1010 to turn (as described in relation to FIG. 6), the flux lines 110 change considerably in magnitudes and orientation. Embodiments of the present invention use an array of magnetic sensors, such as Hall effect sensors, to receive information about the changing magnetic field characteristics and determine parameters which aid the use and function of the external adjustment device 700, and more importantly, of the distraction device 1000 itself. The first parameter is the general proximity of the external magnets 706, 708 of the external adjustment device 700 to the internal permanent magnet 1010 of the distraction device 1000. It is desired that the external magnets 706, 708 of the external adjustment device 700 be placed close enough to the internal permanent magnet 1010 of the distraction device 1000 so that it will function. A goal of the system may be to maximize the torque that the external magnets 706, 708 impart on the internal permanent magnet, and thus to maximize the distraction force delivered by the distraction device 1000. The second parameter is an estimation of the distance between the external adjustment device 700 and the distraction device 1000, particularly the distance between the external magnets 706, 708 of the external adjustment device 700 and the internal permanent magnet 1010 of the distraction device 1000. This distance estimation, as will be explained in greater detail, can be used in estimating the subsequent parameters. The third parameter is the estimated variable dimension of the distraction device 1000, such as distraction length. On some types of adjustable implants, the variable dimension may be length. On other types of adjustable implants (for example, in a restriction device), the adjustable parameter may be diameter or circumference. The fourth parameter is distraction force. Distraction force may be a useful parameter in scoliosis, in particular because in growing patients increased tensile loads on the skeletal system can accelerate growth. This is known as the Heuter-Volkmann principle. Distraction force is also useful in clinical applications concerned with increasing the length of a bone, or changing the angle or rotational orientation of a bone. Again, depending on the implant, the fourth parameter may incorporate other forces, for example, compression force in an adjustable compression implant, for example in trauma applications, such as those disclosed in U.S. Pat. No. 8,852,187. In other medical applications using an adjustable medical implant, it may be useful to know the moment applied on a body part instead of, or as well as, the force applied. For example, in a scoliosis curve, an “un-bending moment” describes the moment placed by a distraction device on the curve to cause it to straighten. For a particular force value, this moment will vary, depending on how far the distraction device is located laterally from the apex of the scoliosis curve. If the lateral distance is known, for example via an X-ray image, the un-bending moment may be calculated from determining the force applied.

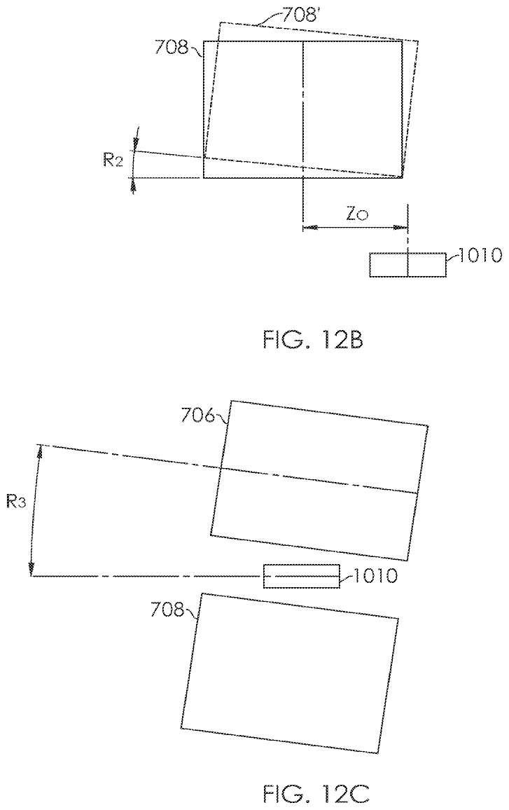

Determining the optimal positioning of the external adjustment device 700 is not always possible. Of course, the implanted distraction device 1000 is not visible to the operator of the external adjustment device 700, and using x-ray imaging to determine its exact location may be difficult, and undesirable due to the additional radiation. Even with an x-ray image that defines a location for the implanted distraction device 1000, the placement of the external adjustment device 700 in a desired location adjacent the skin of the patient may be complicated by extreme curvature of the surface of the patient's body (for example, in scoliosis patients with significant deformity in the torso), or by varying thickness of muscle and fat around the skeletal system (for example circumferentially around the femur in a limb-lengthening patient). FIG. 12A shows, in Cartesian form, the centerline 106 of the external adjustment device 700 aligned with the Y-axis and a gap G between a tangent 707 with the outer surface of the external adjustment device 700 and a tangent 709 with the outer surface of the distraction device 1000. The distance between external magnets 706, 708 and internal permanent magnet 1010 may be slightly larger than the gap G because of their locations within the external adjustment device 700 and the distraction device 1000, respectively (i.e., the housings add slightly to gap G). As the external magnets 706, 708 are placed closer to the internal permanent magnet 1010 of the distraction device 1000, the distraction force that can be generated increases. A lateral offset in alignment is represented by X_{O} along the x-axis, between the centerline 106 of the external adjustment device 700 and the center of the internal permanent magnet 1010. In an embodiment wherein the external adjustment device 700 has only one external magnet, the lateral offset would be represented by the distance between the center of the external magnet and the center of the internal permanent magnet 1010, along the x-axis. In many cases, a smaller X_{O}, allows a higher maximum possible distraction force. Also shown in dashed lines is an external adjustment device 700′ which has been tipped by an angle R₁, causing the external magnet 706′ to be farther from the internal permanent magnet 1010, than if R₁ was close to zero.

FIG. 12B is similar to FIG. 12A, but FIG. 12B shows a side view of the external adjustment device 700 and internal permanent magnet 1010, with the z-axis left to right and the y-axis up and down. An axial offset Z_{O} is drawn between the axial center of the external magnet 708 and the axial center of the internal permanent magnet 1010. Also shown is an alternative configuration, with external magnet 708′ tipped at an angle R₂. The axial offset Z_{O} would tend to lower the maximum possible distraction force. FIG. 12C is a top view that shows a third tipped angle R₃, between the external magnet 706 and the internal permanent magnet 1010. Though in clinical use, R₂ and R₃ are almost always a non-zero magnitude, the larger they are, the lower the potential coupling torque, and therefore the lower the potential distraction force.

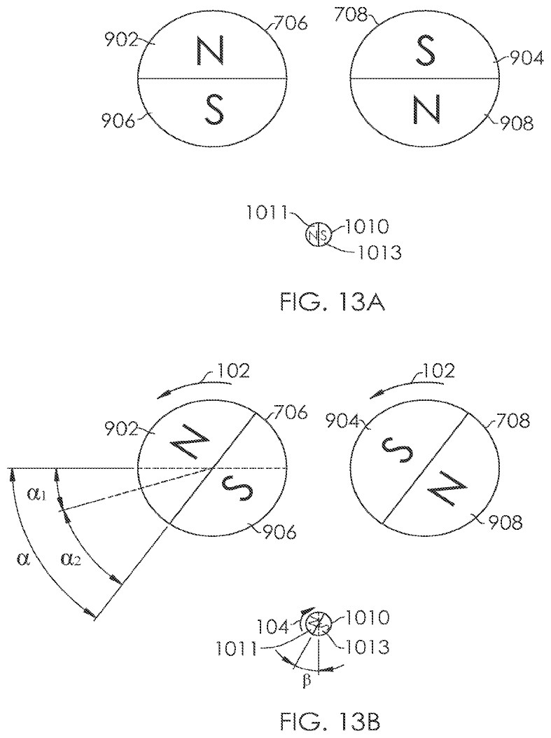

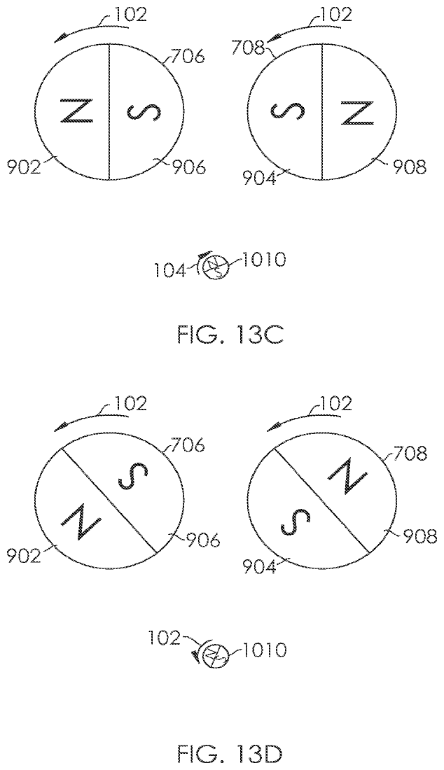

FIGS. 13A through 13D illustrate a variance of magnetic couplings between external magnets 706, 708 and the internal permanent magnet 1010 during an adjustment procedure. FIG. 13A shows a zero torque condition, which may exist, for example, prior to initiating the rotation of the external magnets 706, 708, or at the very start of the operation of the external adjustment device 700. As shown, the north pole 902 of external magnet 706 is pointing in the positive y-direction and the south pole 906 of external magnet 706 is pointing in the negative y-direction, while the south pole 904 of the external magnet 708 is pointing in the positive y-direction and the north pole 908 of the external magnet 708 is pointing in the negative y-direction. The north pole 1011 of the internal permanent magnet 1010 is attracted to the south pole 906 of the external magnet 706 and thus is held in substantially the negative x-direction, and the south pole 1013 of the internal permanent magnet 1010 is attracted to the north pole 908 of the external magnet 708 and thus is held in the positive x direction. All magnets 706, 708, 1010 are in a balanced state and are not fighting each other. As the external adjustment device 700 is operated so that the external magnets 706, 708 begin to turn (as shown in FIG. 13B), it is often the case that there is a nominal resistance torque on the mechanism that is rotatably holding the internal permanent magnet 1010. For example, friction on pins or axles, or friction between the lead screw and the nut of the distraction mechanism. In this particular explanation, it is assumed that external adjustment device either has a single external magnet 706, or has two or more external magnets 706, 708 that rotate synchronously with one another (though other embodiments are possible), and so the reference will currently be made only to the external magnet 706 for simplicity's sake. As external magnet 706 is turned in a first rotational direction 102, up until a first angle α₁, it has not yet applied a large enough applied torque τ_{A} on the internal permanent magnet 1010 to cause it to initiate rotation in a second opposite rotational direction 104. For example, when the applied torque τ_{A} is less than the static threshold resistance torque τ_{ST} of the internal permanent magnet 1010. However, when angle α₁ is exceeded, the applied torque τ_{A} becomes greater than the static threshold torque τ_{ST} of the internal permanent magnet 1010, and thus the rotation of the internal permanent magnet 1010 in the second rotational direction 104 begins, and continues while the external magnet 706 rotates through angle α₂. Thus, when the external magnet 706 reaches angle α (α=α₁+α₂), the internal permanent magnet 1010 has rotated an angle θ, wherein angle β is less than angle α. Angle β is less than or equal to angle α₂. Angle β is less than angle α₂ in cases where the dynamic resistance torque τ_{DR} increases as the internal permanent magnet 1010 rotates through angle β.

FIG. 13C illustrates the orientation of the magnets 706, 708, 1010 after additional rotation has occurred, and as the dynamic resistance torque TDR has increased. This typically occurs as the distraction force of the distraction device 1000 increases, because of increasing friction within the mechanisms of the distraction device 1000, and can occur during the first rotation, or after several rotations. Thus, as seen in FIG. 13C, internal permanent magnet 1010 has rotated a smaller additional amount than the external magnet 706. The term phase lag is used to describe the difference in rotational orientation between the external magnet 706 and the internal permanent magnet 1010. As the dynamic resistance torque τ_{DR} increases, the phase lag increases. The phase lag between the north pole 902 of the external magnet 706 and north pole 1011 of the internal permanent magnet 1010 in the zero torque condition illustrated in FIG. 13A would be defined as 90°. However, for the purposes of the embodiments of the present invention, phase lag is defined as being 0° at the zero torque condition of FIG. 13A. Regardless of the method chosen to define phase lag, the important factor is the change in the phase lag over time (or over the number of rotations). As the dynamic resistance torque τ_{DR} increases even further, a point is reached wherein the dynamic resistance torque τ_{DR} becomes higher than the applied torque TA. This creates a slip condition (or stall condition) wherein the engaged poles of the external magnet(s) and the internal permanent magnet slip past each other, or lose their magnetic engagement. Thus the external magnets 706, 708 of the external adjustment device 700 are no longer able to cause the internal permanent magnet 1010 to rotate. Just prior to slippage the phase lag can be as much as 90°. At the point of slippage, as the poles slip over each other, the internal permanent magnet 1010 typically suddenly and quickly rotates backwards in rotational direction 102 (opposite the rotational direction 104 that it had been turning) at some angle less than a full turn. This is shown in FIG. 13D.

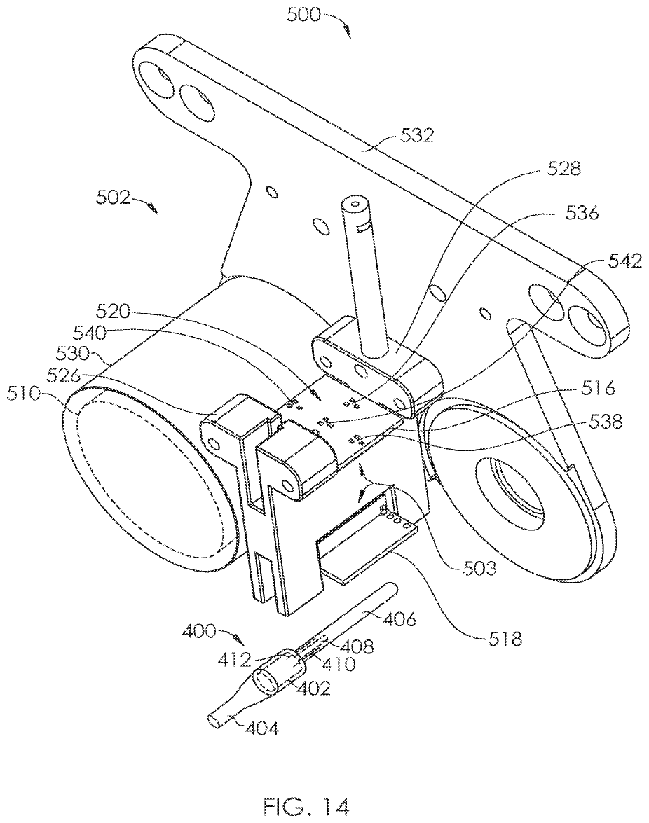

An intelligent adjustment system 500 is illustrated in FIG. 14, and comprises an external adjustment device 502 having a magnetic sensor array 503 which is configured to adjust an adjustable medical device 400 comprising a first portion 404 and a second portion 406, adjustable in relation to the first portion 404. The adjustable medical device 400 is non-invasively adjustable, and contains a rotatable permanent magnet 402, for example a radially-poled cylindrical permanent magnet. The adjustable medical implant 400 is configured to apply an adjustable force within the body. The permanent magnet 402 may be rotationally coupled to a lead screw 408 which is configured to engage with a female thread 410 within the second portion 406, such that the rotation of the permanent magnet 402 causes the rotation of the lead screw 408 within the female thread 410, thus moving the first portion 404 and the second portion 406 longitudinally with respect to each other. The permanent magnet 402 may be non-invasively rotated by applying a torque with one or more external magnets 510 (or 511 of FIG. 16) of the external adjustment device 502. The adjustable medical device 400 is configured for implantation within a patient, and as depicted, is further configured so that the first portion 404 may be coupled to the patient at a first location and the second portion 406 may be coupled to the patient at a second location. In some embodiments, the adjustable medical device 400 may be non-invasively adjusted to increase a distraction force between the first location and the second location. In some embodiments, the adjustable medical device 400 may be non-invasively adjusted to decrease a distraction force between the first location and the second location. In some embodiments, the adjustable medical device 400 may be non-invasively adjusted to increase a compression force between the first location and the second location. In some embodiments, the adjustable medical device 400 may be non-invasively adjusted to decrease a compression force between the first location and the second location. In some embodiments, the adjustable medical device 400 may be non-invasively adjusted to perform two or more of these functions. Alternatively, the adjustable medical device may be a restriction device, configured to be adjusted to increase or decrease a diameter. For example, a diameter that at least partially restricts a body conduit, such as a blood vessel, a gastrointestinal tract or a urinary tract. In an embodiment of this nature, the movement of the first portion 406 in relation to the second portion 406 may increase or decrease traction or tension on a cable or tension member, which in turn causes the restriction (or increase, as the case may be) in diameter of the restriction device.

The magnetic sensor array 503 may comprise two circuit boards 516, 518, for example printed circuit boards (PCBs). The first circuit board 516 may be located in opposition to the second circuit board 518. For example, the first circuit board 516 may be located above and generally parallel to the second circuit board 518. Each circuit board 516, 518 may have a subarray 520 of magnetic sensors 536, 538, 540, 542, for example, Hall effect sensors. A second external magnet 511 (FIG. 16) or even more external magnets may be disposed on the external adjustment device 502. In FIG. 14, a second external magnet 511 has been removed to show detail of the magnetic sensor array 503. Standoff blocks 526, 528 may be disposed on the external adjustment device 502 to hold the first and second circuit boards 516, 518 in place. The standoff blocks 526, 528 may be movable in one or more directions to allow fine adjustment of multiple dimensions of each circuit board 516, 518, as needed, to tune the magnetic sensor array 503. The one or more external magnets 510 are rotatably secured to a base 532, and may be covered with a stationary cylindrical magnet cover 530. It may be desired to rotatably secure the one or more external magnets 510 to the base well enough so that they do not vibrate or rattle, thereby advantageously increasing the signal to noise ratio of the magnetic sensors and the overall effectiveness of the sensor array 503.

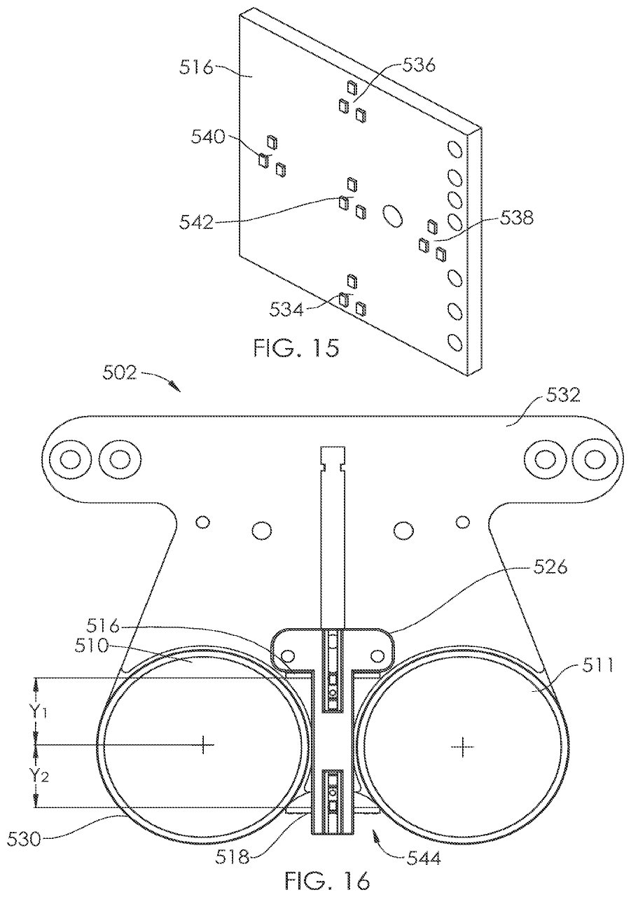

The circuit boards 516, 518 may be substantially identical to each other, or may be mirror images of each other. FIG. 15 shows circuit board 516 in more detail. Five Hall effect sensors (HES) include a forward HES 534, a back HES 536, a left HES 538, a right HES 540, and a middle HES 542 (herein, forward HES 534, back HES 536, and middle HES may be referred to, individually or collectively, as center HES). In FIG. 14 circuit board 516 is shown having the Hall effect sensors 534, 536, 538, 540, 542 extending upward, while the circuit board 518 is shown having its Hall effect sensors extending downward (not visible in FIG. 14). In some embodiments, it may be advantageous to have the HES of circuit board 518 extending downward to minimize the distance between the Hall effect sensors and the permanent magnet 402. In some embodiments, circuit board 518 may thus have a mirror image to circuit board 516, so that the left HES 538 of circuit board 516 is directly above the left HES of circuit board 518, etc. However, if the Hall effect sensor used for the left HES is identical to the Hall effect sensor used for the right HES, and the same for forward HES and back HES, the same circuit board may be used for both circuit boards 516, 518, thus reducing manufacturing costs. It is envisioned that printed circuit boards (PCBs) would be used to allow conductive tracks for connections to a voltage source (for example, +5 Volts) for each Hall effect sensor.

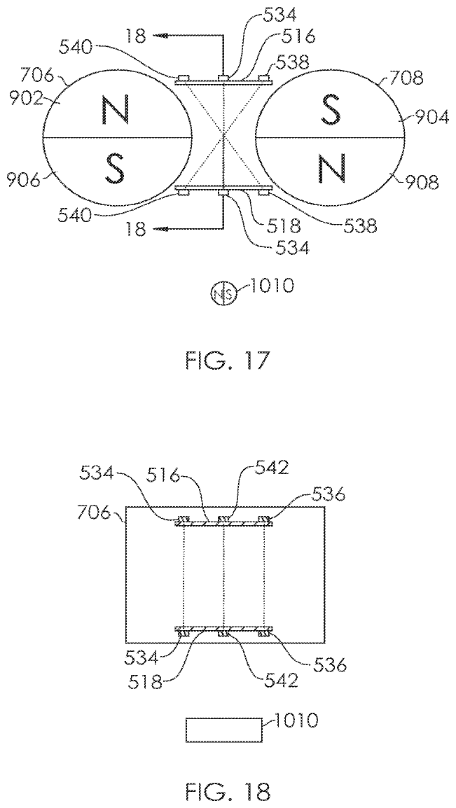

In some embodiments, the Hall effect sensors 534, 536, 538, 540, 542 comprise linear Hall effect sensors. The configuration of the circuit boards 516, 518 (i.e., one above the other) aids their use in differential mode, as will be described in regard to FIG. 17. Because the middle HES 542, in both circuit boards 516, 518, is the furthest of the Hall effect sensors from the external magnets 510, 511, it can be less prone to saturation. Therefore, in such embodiments, a more sensitive Hall effect sensor may be used as the middle HES 542. For example, an A1324, produced by Allegro Microsystems LLC, Irvine, Calif., USA, which has a sensitivity of between about 4.75 and about 5.25 millivolts per Gauss (mV/G), or more particularly 5.0 mV/G, may be used. For the other Hall effect sensors (e.g., 534, 536, 538, 540), which are located closer to the external magnets 510, 511 and more likely to be saturated, a less sensitive Hall effect sensor may be used. For example, an A1302, also produced by Allegro Microsystems LLC, Irvine, Calif., USA, with a sensitivity of about 1.3 mV/G may be used.

Turning to FIG. 16, the orientation of each circuit board 516, 518 is shown in relation to the centers of each external magnet 510, 511. An exemplary arrangement comprises external magnets 510, 511 having diameters between about 2.54 cm (1.0 inches) and 8.89 cm (3.5 inches), and more particularly between about 2.54 cm (1.0 inches) and 6.35 cm (2.5 inches). The length of the external magnets 510, 511 may be between about 3.81 cm (1.5 inches) and 12.7 cm (5.0 inches), or between about 3.81 cm (1.5 inches) and 7.62 cm (3.0 inches). In a particular embodiment, the external magnets have a diameter of about 3.81 cm (1.5 inches) and a length of about 5.08 cm (2.0 inches), and are made from a rare earth material, such as Neodymium-Iron-Boron, for example using a grade greater higher N42, greater than N45, greater than N50, or about N52. Returning to FIG. 14, exemplary sizes for the permanent magnet 402 may include a diameter between about 6.35 mm (0.25 inches) and 8.89 mm (0.35 inches), between about 6.85 mm (0.27 inches) and 8.13 mm (0.32 inches), or about 7.11 mm (0.28 inches). The permanent magnet 402 may have a length of between about 1.27 cm (0.50 inches) and 3.81 cm (1.50 inches), between about 1.77 cm (0.70 inches) and 3.18 cm (1.25 inches), or about 1.85 cm (0.73 inches), or about 2.54 cm (1.00 inches). In a particular embodiment, the permanent magnet 402 may be made from a rare earth material, such as Neodymium-Iron-Boron, for example using a grade greater higher N42, greater than N45, greater than N50, or about N52.

Turning again to FIG. 16, circuit board 516 (also called upper circuit board) may be located a distance Y₁ from the center of the external magnets 510, 511 of about 15 mm to 32 mm, or about 21 mm. Circuit board 518 (also called lower circuit board) may be located a distance Y₂ from the center of the external magnets 510, 511 of about 17 mm to 35 mm, or about 26 mm. The external adjustment device 502 may include a depression 544 between the two external magnets 510, 511 to allow skin and/or fat to move into the depression when the external adjustment device is pressed down on the patient, thereby allowing the external magnets 510, 511 to be placed as close as possible to the permanent magnet 402. In some embodiments of external adjustment devices 502 having two external magnets 510, 511, the central axes of the two external magnets 510, 511 may be separated from each other by between about 50 mm and 100 mm, between about 55 mm and 80 mm, or about 70 mm.

In FIG. 17 a front view of the external adjustment device 502 (of FIGS. 14 & 16) shows the pairs of Hall effect sensors that are coupled to the same differential amplifier. The left HES 538 of circuit board 516 is paired with the right HES 540 of circuit board 518. The left HES 538 of circuit board 518 is paired with the right HES 540 of circuit board 516. In FIG. 18, the forward HES 534 of circuit board 516 is paired with the forward HES 534 of circuit board 518. The middle HES 542 of circuit board 516 is paired with the middle HES 542 of circuit board 518. And, the back HES 536 of circuit board 516 is paired with the back HES 536 of circuit board 518. Dotted lines have been drawn in in both FIGS. 17 and 18 to better illustrate the pairings.

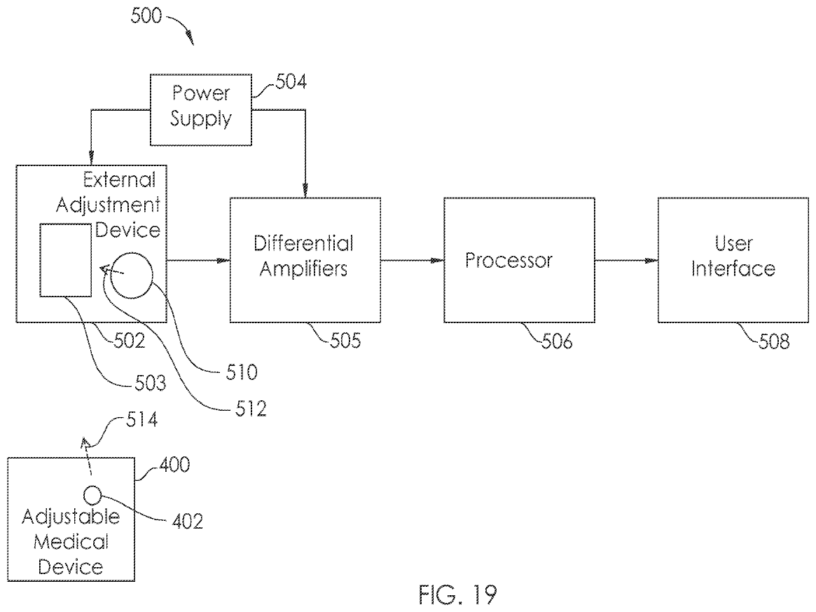

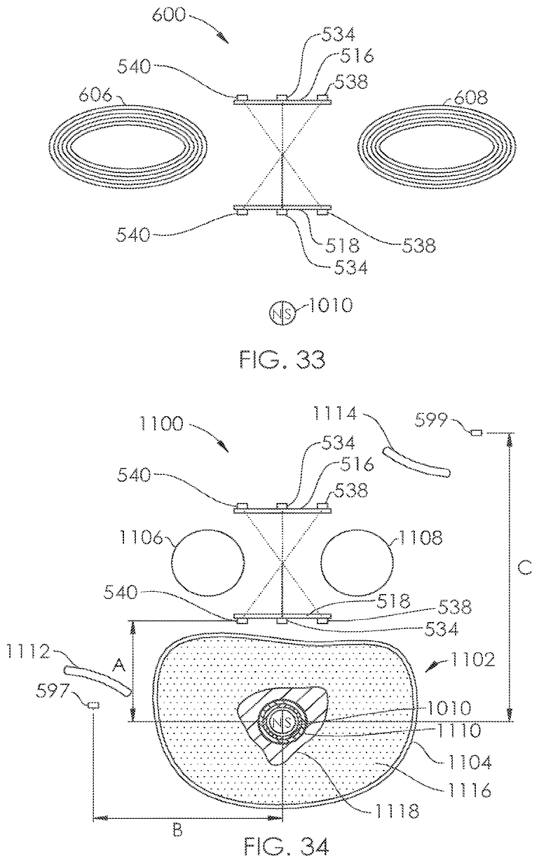

In FIG. 19, an external adjustment device 502 having a sensor array 503 and having at least one external magnet 510 configured for rotation is powered by a power supply 504. This power supply 504 (or a separate power supply) powers differential amplifiers 505, to which the Hall effect sensors (534, 536, 538, 540, 542 of FIGS. 17 and 18) are coupled. The at least one external magnet 510 of the external adjustment device 502 is rotated (e.g., by a motor 840 of FIG. 4) and magnetically couples to the permanent magnet 402 of the adjustable medical device 400. The coupling between the at least one external magnet 510 and the permanent magnet 402 may have variable coupling and torque characteristics (e.g., increasing dynamic resistance torque τ_{DR}) which cause a varying magnetic field represented by components (i.e., vectors) 512 and 514. It should be mentioned that it is still within the scope of the present invention that embodiments could be constructed so that the one or more rotatable external magnet(s) 510, 511 are one or more electromagnets, creating rotatable magnetic fields comparable to, for example, those created by two rotatable permanent magnets. FIG. 33 illustrates an external adjustment device 600 comprising two electromagnets 606, 608 for creating rotatable magnetic fields. The external adjustment device 600 is otherwise similar to the external adjustment device 502 of FIGS. 14-19. Returning to FIG. 19, a processor 506 (for example a microprocessor) processes signals from the differential amplifiers 505, and the resulting information is displayed on a user interface 508.

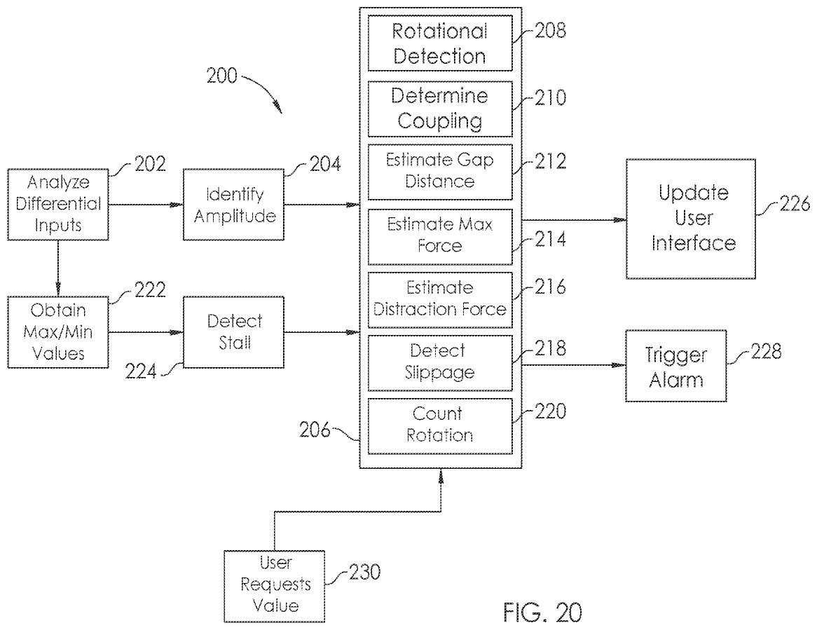

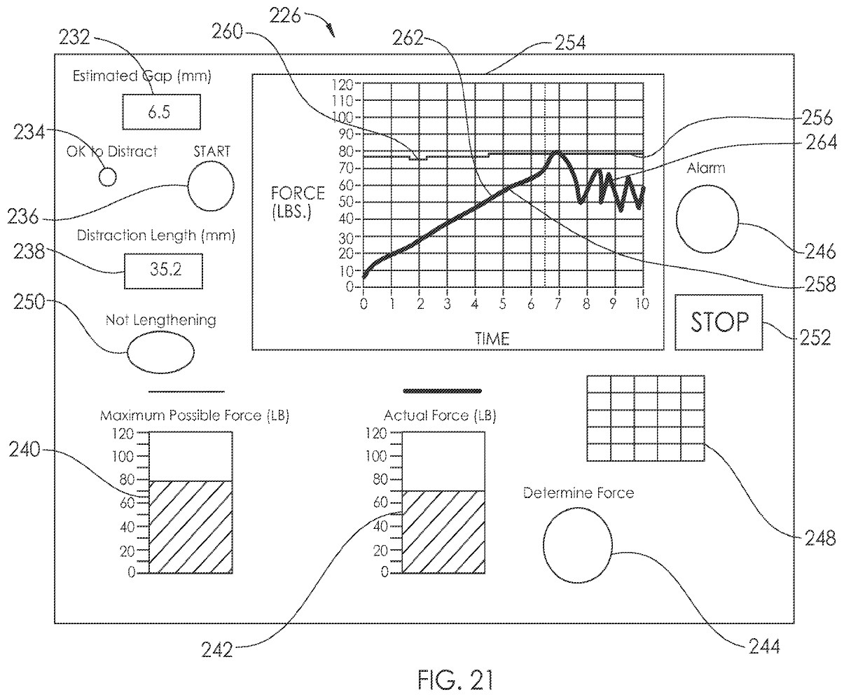

FIG. 20 illustrates the system logic 200 within an intelligent adjustment system, (e.g., 500 of FIG. 14) that allows it to take signals received by the sensor array 503 and determine or estimate: 1) the general proximity of the external magnets 706, 708, 510, 511 of the external adjustment device 700,502 to the internal permanent magnet 1010, 402 of the distraction device 1000, 400, 2) a distance between the external adjustment device 700,502 and the distraction device 1000, 400, particularly the distance between the external magnets 706, 708, 510, 511 of the external adjustment device 700, 502 and the internal permanent magnet 1010, 402 of the distraction device 1000, 400, 3) the estimated distraction length of the distraction device 1000, 400, and 4) the distraction force. Data is acquired, in continuous mode in some embodiments, and, for example, at a sampling rate of 1,000 Hz. At block 202 differential inputs from the middle HES 542, left HES 538, and right HES 540 are analyzed, with the maximum and minimum values (voltages) of each complete rotation cycle, thus at block 204, identifying the amplitude of the waveform of the middle HES 542. This amplitude will be used during several subsequent functions performed in the blocks outlined/encircled by block 206. At block 208, rotational detection is performed. For example, in one embodiment, if the amplitude of the waveform is smaller than 4.2 Volts, then the permanent magnet 1010, 402 of the distraction device 1000, 400 is determined to be rotationally stationary. At block 210, the general proximity of the external adjustment device 700, 502 to the permanent magnet 1010, 402 of the distraction device 1000, 400 is determined. For example a yes or no determination of whether the external adjustment device 700, 502 is close enough to the permanent magnet 1010, 402 to allow operation of the external adjustment device 700, 502. In one embodiment, the data acquisition array is analyzed and if the first and last elements (i.e., all of the values measured in the data acquisition array) are smaller than 0.5 Volts, then the peak of the waveform produced by the Hall effect sensors is complete for being processed. If the amplitude of the waveform is larger than 9.2 Volts, the external adjustment device 700, 502 is acceptably close to the permanent magnet 1010, 402 of the distraction device 1000, 400 to warrant continued adjustment, without aborting.

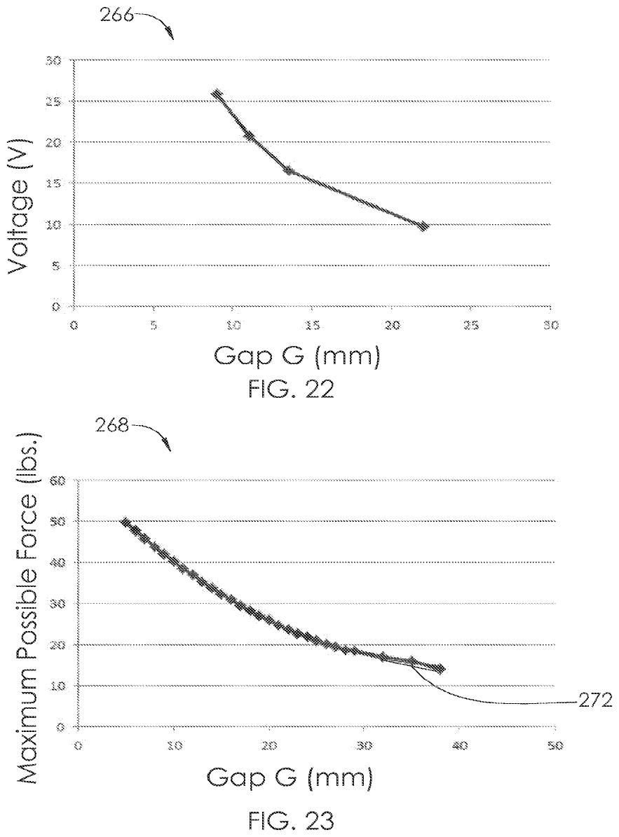

At block 212, an estimation is done of the actual distance between the external adjustment device 700, 502 and the distraction device 1000, 400 (or between the external magnets 706, 708, 510, 511 and the permanent magnet 1010, 402). Empirical data and curve fit data are used to estimate this distance (gap G). For example, for one particular embodiment, FIG. 22 illustrates a graph 266 of empirical data obtained of voltage (V) for a series of gaps G. A curve fit generated the equation:

V=286.71×G^{-1.095}

where V is voltage in Volts, and G is gap G in millimeters.

Returning to FIG. 20, at block 214 the maximum distraction force at the current distance (gap G) is estimated based on empirical data and curve fit data. For example, for one particular embodiment, FIG. 23 illustrates a graph 268 of maximum possible force in pounds (lbs.) for a series of gaps G. A curve fit 272 generated the equation:

F=0.0298×G²−2.3262×G+60.591

where F is Force in pounds (lbs.), and G is gap G in millimeters

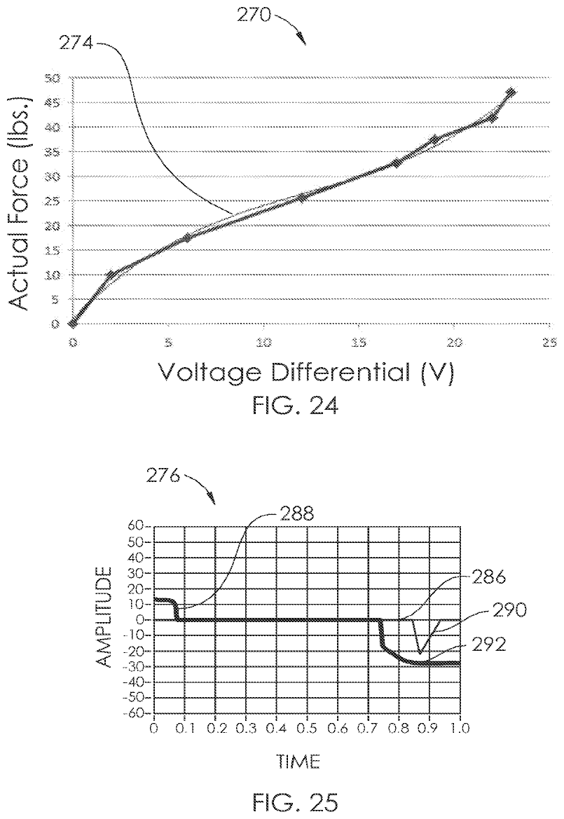

Returning to FIG. 20, at block 216 a real time estimate of distraction force is performed based on empirical data and curve fit data. For example, for one particular embodiment, FIG. 24 illustrates a graph 270 of estimated or actual distraction force in pounds (lbs.) over a range of voltage differentials. A curve fit 274 generated the equation:

F=0.006×V_{d}³−0.2168×V_{d}²+3.8129×V_{d}+1.1936

where F if Force in pounds (lbs.), and V_{d} is differential voltage in Volts.

Returning to FIG. 20, a button may be pushed on a user interface 226, whenever a value for this force is desired, or it may be set to continually update. At block 218, slippage between the external magnets 706, 708, 510, 511 and the permanent magnet 1010, 402 is detected. First, at block 222, the differential input between the left and right HES 538, 540 is acquired, and the maximum and minimum values obtained. Then, at block 224, stall detection logic is run. In one embodiment, if the ratio between the maximum and minimum values of the waveform between two periods is larger than 0.77 Volts during a valid waveform period, and if it happens two times in a row, the slippage is detected (for example, between the left HES 538 of circuit board 516 and the right HES 540 of circuit board 518 and/or between the right HES 40 of circuit board 516 and the left HES 538 of circuit board 518). In one particular embodiment, if the current amplitude is 1.16 times (or more) larger than the previous current amplitude (or 1.16 times or more smaller), slippage is detected. In one embodiment, if the difference between the maximum index and the minimum index is smaller than 12 Volts, slippage is detected. If a stall is detected by the left and right HES 538, 540, slippage is detected. If slippage is detected, an alarm 228 may be sounded or lit.

Referring now to FIG. 25, a graph 276 is illustrated of two differential voltages over time in an embodiment of the present invention. A differential voltage may be the measured difference in voltage potential between two associated hall-effect sensors on external adjustment device 700. For example, a differential voltage may be the measured difference in voltage potential from a middle pair of hall-effect sensors 534 (alternatively any of the center HES, including 534, 542, and 536), which are comprised of a hall-effect sensor at the top of the magnets in external adjustment device 700 and a hall-effect sensor at the bottom of the magnets in external adjustment device 700, the bottom hall-effect sensor in line with the top hall-effect sensor. The bottom hall-effect sensor of the pair of hall-effect sensors 534 may have a measured voltage that includes voltage due to the magnetic field of distraction device 1000 (alternatively any of the corresponding center HES, including 534, 542, and 536). However, it may be desirable to subtract out from that measured voltage any influence from the magnets of external adjustment device 700. This subtraction can be done using the measured voltage of the top hall-effect sensor in the pair of hall-effect sensors 534, because the top hall-effect sensor may be too far away from the distraction device 1000 for the magnetic field of distraction device 1000 to have a significant impact on the measured voltage of the top hall-effect sensor. The top hall-effect sensor primarily measures voltage due to the magnetic fields of the magnets in external adjustment device 700. Thus, by determining the differential voltage of the measured voltages of the pair of hall-effect sensors 534, the voltage due to the magnetic field of the distraction device 1000 can be determined. In graph 276, a differential voltage 286 (thin line) may be a graph of the differential voltage between the middle HES pair 542 of circuit board 516 and 542 of circuit board 518, and it may be used to calculate many of the parameters or estimates using the systems disclosed herein. Differential voltage 286 may have a triangular perturbation 290. The triangular perturbation 290 is typically located within the cycle of the differential voltage 286. Changes in the amplitude of the triangular perturbation 290 may represent, for example, slippage or may represent the changes in coupling torque. The external adjustment device 700 may be configured to determine when slippage is occurring or when it is coupled to the distraction device 1000 by evaluating the triangular perturbation 290 occurring in differential voltage 286. In graph 276, a differential voltage 288 (thick line) may be a graph of the differential voltage between side pairs (for example, between the left HES 538 of circuit board 516 and the right HES 540 of circuit board 518) of hall-effect sensors, and may be used for confirmation of magnetic slippage occurring between the external adjustment device 700 and the distraction device 1000. Differential voltage 288 may have a perturbation 292. Perturbation 292 is typically located within the cycle of the differential voltage 288. Changes in the amplitude of the perturbation 292 may occur during magnetic slippage. The external adjustment device 700 may be configured to determine when slippage is occurring by evaluating the perturbation 292 occurring in differential voltage 288.

Returning to FIG. 20, at block 230, when a real time torque value is requested (for example, but pushing a button on the user interface 226), the voltage or amplitude of the waveform is recorded. At block 220, the rotation cycles are counted (this may occur continuously). The distraction length is also counted. For example, in one embodiment, 0.32 mm of linear distraction occurs for every rotation of the internal permanent magnet 1010, 402. In another embodiment, 0.005 mm of linear distraction occurs for every rotation of the internal permanent magnet 1010, 402. The number of rotations may be the number of rotations of the internal permanent magnet 1010, 402 or a fraction or multiple of the number of rotations of the internal permanent magnet 1010, 402 (i.e., “rotations” can be a non-integer number and can be less than 1 or greater than 1). For example, in a distraction device 1000, 400 having a gear module 412 (FIG. 14) between the internal permanent magnet 1010, 402 and the lead screw 408, it may be desired to count the number of rotations of the internal permanent magnet 1010, 402 divided by the gear reduction. For example in a gear reduction of 64:1 wherein the lead screw 408 rotates at a number of rotations per unit time that is 1/64 times that of the internal permanent magnet 1010, 402, the number counted by the system 500 may be the number of rotations of the internal permanent magnet 1010, 402 divided by 64.

Measuring distraction length of a distraction device 1000 may be both a function of measuring slippage between the external adjustment device 700 and distraction device 1000, as well as measuring the rotation of the magnets 706, 708 within the external adjustment device 700. If the external adjustment device 700 and the distraction device 1000 are coupled and no slipping or stalling is occurring, the internal permanent magnet 1010 of the distraction device 1000 is presumed to be rotating. The rotation of the magnets 706, 708 may be counted by the external adjustment device 700 in order to determine the rotation of the internal permanent magnet 1010 of the distraction device 1000. From the amount of rotations of the internal permanent magnet 1010, the distraction length can be inferred from the process described above since the dimensions and properties of the distraction device 1000, magnets 706, 708, internal permanent magnet 1010, and gear module 412 are known before-hand. Thus, the distraction length of the distraction device 1000 can be backed out or calculated from the number of rotations of magnets 706, 708 of the external adjustment device.

The above process relies on the assumption that the lead screw 408 of the distraction device 1000 rotates as the internal permanent magnet 1010 is rotated. However, this may not necessarily be true. For example, if the gear module 412 and/or any intermediary member (such as a coupling pin) is broken, then the internal permanent magnet 1010 may not be mechanically coupled to the lead screw 408. Rotating the internal permanent magnet 1010 would not also rotate the lead screw 408. In this situation, there would be no resistance force. The external adjustment device 700 may be unable to determine the difference between this scenario where the coupling pin is broken (and no lead screw 408 rotation is occurring) and a normal scenario where the lead screw 408 is being freely rotated against zero resistance force. In both scenarios the resistance force may be approximately zero. Thus, it may not be immediately obvious to the user that the lead screw 408 is uncoupled and not rotating since the presence of zero resistance force may arise in normal usage. This is merely one example for which the measured or estimated distraction length of the distraction device 1000 could be considerably different from the actual distraction length of the distraction device 1000.

To remedy this problem, the software of the external adjustment device 700 may be configured with algorithms for detecting when the distraction device 1000 is actually being distracted. For example, an algorithm may check to see how long the period of zero resistance force is. If a user is attempting to distract a distraction device 1000 and experiencing zero resistance force over a longer period than is typical for a distraction device 1000 to experience zero resistance force, then the external adjustment device 700 may alert the user, such as through the user interface shown in FIG. 21.

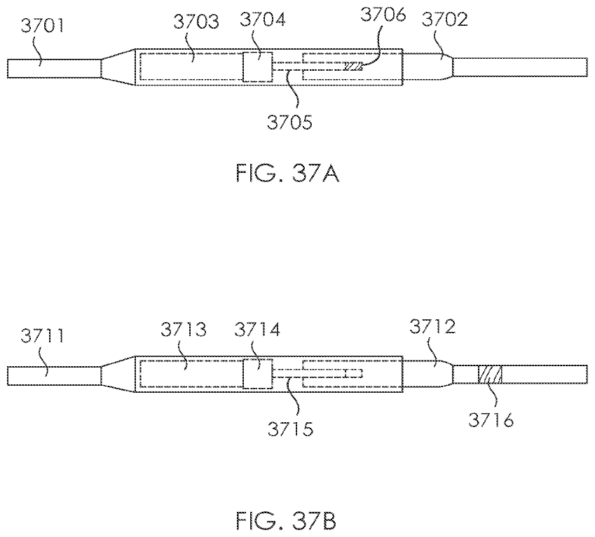

Other methods of addressing this problem may involve alternate embodiments of the distraction device. A magnet could be placed in lead screw 408, so that the amount of rotations of the lead screw 408 can be measured directly and independent of having a functional (e.g., not broken) coupling pin. A magnet could also be placed in the second portion 406 of the distraction device 400, the portion otherwise known as the distraction rod that has a threaded recess with which the lead screw 408 may mate. The magnet could be placed at any point in the distraction rod, and the external adjustment device 700 could be configured to measure the distance between the internal permanent magnet 402 and the magnet in the distraction rod to calculate the actual distraction distance since the dimensions of each portion of the distraction device 400 can be known before-hand. A more in-depth discussion of these methods is provided in the descriptions of FIGS. 37A, 37B, 38 and 39. In addition to the functions described that are possible with the magnetic sensor array 503, it is possible to use the magnetic sensor array 503 in place of the Hall effect sensors 924, 926, 928, 930, 932, 934, 936, 938 of the embodiments described in relation with FIGS. 7-10B in order to track rotation of the external magnet(s) 706, 708,510, 511.

One embodiment of a user interface 226 for conveying information to the user and receiving inputs from the user is illustrated in FIG. 21. In FIG. 21, the user interface 226 may comprise a graphic user interface (GUI) and may include a display and control buttons, or one or more touchscreens. The user interface may include an estimated gap display 232, which tells the user the approximate distance (the gap distance) between the external adjustment device 700 and the distraction device 1000, or the approximate distance between the external magnets 706, 708 of the external adjustment device 700 and the internal permanent magnet 1010 of the distraction device 1000. The gap distance may be measured using any of a variety of methods. For example, medical imaging devices or systems may be used to determine the distance between a distraction device 1000 implanted in a patient to the top layer of skin at which the external adjustment device 700 would be applied in order to rotate distraction device 1000. In some embodiments, this gap distance may be the only user input required to use the external adjustment device 700. For a given gap distance, the relationship between the measured voltage potential from the Hall effect sensors of the external adjustment device 700 and the actual force being applied to the distraction device 1000 may be known. That relationship may be pre-determined for the given gap distance and used going forward to infer the actual force being applied to the distraction device 1000, which may then be reported to the user through the user interface. If the gap distance, is within an appropriate range that the external adjustment device 700 may exert sufficient force on the distraction device 1000, an “OK to distract” indicator 234 may light up, vibrate, or sound, depending on whether it is a visual (e.g., LED), tactile, or audio indicator. This may inform the user that the gap distance is within the operating range of the external adjustment device 700. More discussion of the gap distance is provided below.

At this point, the user may initiate distraction/retraction of the distraction device 1000 by pressing a “Start” button 236 of the external adjustment device 700. Alternatively, neither the “OK to distract” indicator 234 nor the “Start” button 236 may appear on the user interface 226 until the gap distance is determined to be within an acceptable level, and only then the “Start” button 236 will be displayed on the user interface 226. For example, in one embodiment, an acceptable gap distance is a distance below which a coupling may be generated between the external magnets 706, 708 of the external adjustment device 700 and the internal permanent magnet 1010 of the distraction device 1000 sufficient to generate a significant distraction force (e.g., enough to distract bones, joints or tissue). In some embodiments, this may be a gap distance of 51 mm or less. In other embodiments, this may be a gap distance of 25 mm or less. In other embodiments, this may be a gap distance of 12 mm or less. In some embodiments, the significant distraction force to distract bones, joints, or tissue may be 1 pound or greater. In other embodiments, it may be 20 pounds or greater. In other embodiments, it may be 50 pounds or greater. In some embodiments, there may be an additional indicator if the gap distance is too small. For example, if the gap is 1 mm or less, the system 500 may be set to not function, for example, in order to protect components of body tissue from forces or torques that are too large. This feature may function based on data that illustrates the relationship between voltage, force, or torque and the gap distance, example graphs of which may be similar to the graphs shown in FIGS. 22 and 23. A maximum possible force display 240 may indicate the expected maximum possible force at the current condition (i.e., the current gap distance), either graphically as shown, or with the display of a number. This feature may function based on data that illustrates the relationship between force and the gap distance, example graphs of which may be similar to the graph of FIG. 23.

If the “Start” button 236 is pressed and the external adjustment device 700, begins to distract the distraction device 1000 the system 500 may begin counting the revolutions of the internal permanent magnet 1010 and determining the estimated distraction length of distraction device 1000. In other embodiments, the method of determining the distraction length of distraction device 1000 may be different than counting the revolutions of internal permanent magnet 1010 to infer the distraction length. For example, the distraction length may be directly measured. Here, the distraction length of distraction device 1000 is being estimated and may be displayed on the distraction length display 238. An estimated force or actual force display 242 may show the current distraction force (or compression force or other force). This may be updated at any range of update rates. Alternatively, it may be updated only when the user presses a “Determine Force” button 244.

If slippage between magnets 706, 708 of the external adjustment device 700 and internal permanent magnet 1010 of the distraction device 1000 is detected, a “Not Lengthening” indicator 250 may light up, vibrate, or sound, depending on whether it is a visual (e.g., LED), tactile, or audio indicator. This may inform the user that slippage is occurring. Or, it may indicate the breakage of a connector pin. If at any time any significant event occurs for which user should be notified, an alarm 246 may light up, vibrate, or sound, depending on whether it is a visual (e.g., LED), tactile or audio indicator. Such events may include reaching too high of a force, or reaching the limit of the distraction device 1000, such as its maximum or minimum length. Alarm 246 may also alert the user at the same time that slippage is occurring and being signaled by the “Not Lengthening” indicator 250.

A data input module 248 may be used to input data, such as, for example, the starting distraction length of the distraction device 1000, the gap distance that is reflected by gap display 232, the model of the distraction device, and/or any relevant patient demographic data. At any point during the operation of the system 500, the user may press a “Stop” button 252 to stop all activity and prevent the external adjustment device 700 from rotating its magnets 706, 708 such that the distraction device 1000 will not be distracted or retracted.

A graph 254 (FIG. 21) may be included on the user interface 226, which may display to the user the maximum possible force 256 and the actual force 258 over time. The maximum possible force 256 over time shown in graph 254 may have shifts 260 in its graph. Shifts 260 of the maximum possible force 256 over time may be caused by the gap distance changing due to the user applying more or less pressure on the external adjustment device 700, 502. The graph 254 of the user interface 226 may also include a graph of the actual distraction force 258. The graph of the actual distraction force 258 may include a portion in which ramp up 262 occurs. The ramp up 262 portion of the graph may visually represent the period of time in which the external adjustment device 700 is rotating the distraction device 1000 without significant resistance, just prior to when the distraction device 1000 begins to encounter the resistance (e.g., caused by tissue or bone). The graph of the actual distraction force 258 may also include a portion in which slippage jumps 264 occur. The slippage jumps 264 portion of the graph may visually represent the period of time in which slippage is occurring between the external adjustment device 700 and the distraction device 1000. The slippage jumps 264 may be a result of the applied torque τ_{A} on the internal permanent magnet 1010 of distraction device 1000 increasing a little, and then quickly dropping as slippage occurs due to the distraction device 1000 being caught by resistance. The jumps repeat as the magnets of external adjustment device 700 continue spinning and applying torque on the distraction device 1000. The system 500 may have limits that shut down the system if the voltage values demonstrate that the device is being used improperly. In this disclosure, reference to external magnets 706, 708 may be considered to also reference external magnets 510, 511 where appropriate, and vice versa. For example, if a patient were to turn the external adjustment device 502 backwards, and/or to run the external magnets 510, 511 in an incorrect direction, the voltage values may signal the system 500 to shut off and prevent distraction or retraction of distraction device 1000. Thus, the auto shut-down feature may be used to prevent improper or undesired use of internal permanent magnets 1010 and 402, distraction device 1000 and adjustable device 400, and external adjustment devices 700, 502.

Referring back to FIG. 20, the software of the external adjustment device may be further configured so that system 500 may have “smart” functionality that may be carried over into, or implemented through, the user interface of FIG. 21. Data input module 248 of the user interface 226 may allow the user to set limits for rotations or distraction lengths. The system 500 may be configured to automatically stop rotating the magnets in the external adjustment device 700 when a user-set number of rotations of the magnets in the external adjustment device 700 is reached, when the internal magnet 1010 of the implant has met the number of set rotations, or when the implant has been distracted/retracted to have the desired distraction length. Thus, a user may be able to have a desired distraction length for the implant that the external adjustment device automatically adjusts the implant to once coupling is achieved. Similarly, the system 500 may be configured to shut down once a certain force, coupling torque, or differential voltage reading is achieved.

The system 500 may also be able to detect coupling torque based on the force on the implant using the known dimensions and characteristics of the implant. The system 500 may have a database of compatible implants that may be used with the external adjustment device 700. A user may be able to use data input module 248 of the user interface 226 in order to select an implant from the database of compatible implants, which could store dimensions and characteristics of the implant, for example, maximum distraction length and minimum distraction length of the implant (such that it could prevent the user from trying to distract the implant out of that operating range). The system 500 could know the positioning of magnets within the implant, such that the distraction length can be estimated (such as through the methods and embodiments described with respect to FIGS. 37A and 37B). The system 500 could know the various coupling ratios between the rotational magnets of the external adjustment device and the internal magnet of the implant, ratios between the internal magnet of the implant and the gear box of the implant, ratios between the gear box of the implant and the lead screw, and even threading of the lead screw. This could allow system 500 to indirectly estimate distraction length from magnet rotations without requiring any further inputs from the user other than the implant identifier. This sort of information may allow the system to also detect the coupling torque of the implant, which may be reported through user interface 226, such as plotted on graph 254.

The system 500 may be able to detect if the coupling pin of the implant is broken. In some situations, the coupling pin could be broken so that the lead screw of the implant does not rotate when the internal magnet of the implant rotates. In such cases, the external adjustment device could perceive there to be zero resistance force, as if the body of the patient exerts no resistance on the implant, when, in fact, the internal magnet of the implant is spinning within the implant housing. System 500 may be configured to differentiate between a broken coupling pin and a scenario with actual zero resistance force. In a situation when the coupling pin is broken, the user interface 226 may notify the user that the pin is broken. For example, this may be done through Alarm 246. The system 500 may also be configured to shut down and prevent any further rotation of the magnets in external adjustment device when it detects the coupling pin is broken. In addition, options may be provided to a user through user interface 226 in order to override the system 500 determination of whether the coupling pin is broken or not. For example, the user may be able to ignore/override a message or alarm that the pin is broken and subsequently force rotation of the magnets in the external adjustment device (e.g., by pressing and/or holding down Start button 236). This could allow the user to continue using the device even in zero resistance force scenarios that system 500 erroneously determines to be a result of a broken coupling pin.

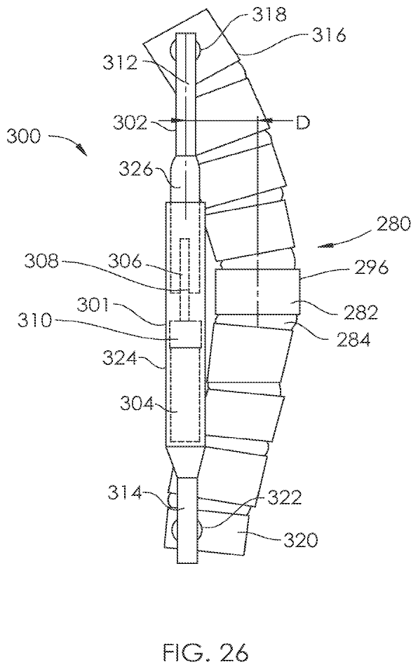

Several embodiments of adjustable implants configured for use with the system 500 are illustrated in FIGS. 26-32. The adjustable spinal implant 300 of FIG. 26, is secured to a spine 280 having vertebrae 282 and intervertebral discs 284. A first end 312 is secured to a portion of the spine 280, for example, to a first vertebra 316 with a pedicle screw 318. A second end 314 is secured to a portion of the spine 280, for example, to a second vertebra 320 with a pedicle screw 322. Alternatively, hooks, wires or other anchoring systems may be used to secure the adjustable spinal implant 300 to the spine 280. Many different portions of the vertebrae may be used to secure the adjustable spine implant 300. For example, the pedicle, the spinous process, the transverse process(es), the lamina, and the vertebral body, for example in an anteriorly placed adjustable spinal implant 300. The adjustable spinal implant 300 may alternatively be secured at either or both ends to ribs, or ilium. The adjustable spinal implant 300 comprises a first portion 301 and a second portion 302. The first portion 301 includes a hollow housing 324 and the second portion 302 includes a rod 326 which is axially extendable in both directions, and which is telescopically contained within the hollow housing 324. A permanent magnet 304 is contained within the hollow housing 324, and is configured for rotation. The permanent magnet 304 is coupled to a lead screw 306 via an intermediate gear module 310. The gear module 310 may be eliminated in some embodiments, with the permanent magnet 304 directly connected to the lead screw 306. In either embodiment, rotation of the permanent magnet 304 (for example, including by application of an externally applied moving magnetic field of an external adjustment device 700, 502) causes rotation of the lead screw 306 (either at the same rotational velocity or at a different rotational velocity, depending on the gearing used). The lead screw 306 is threadingly engaged with a female thread 308, disposed within the rod 326. Certain embodiments of the adjustable spinal implant 300 may be used for distraction of the spine 280 or compression of the spine 280. Certain embodiments of the adjustable spinal implant 300 may be used to correct the spine of a patient with spinal deformity, for example due to scoliosis, hyper (or hypo) kyphosis, or hyper (or hypo) lordosis. Certain embodiments of the adjustable spinal implant 300 may be used to distract a spine, in order to open the spinal canal which may have been causing the patient pain. Certain embodiments of the adjustable spinal implant 300 may be used for adjustable dynamic stabilization of the spine, for control of the range of motion. Certain embodiments of the adjustable spinal implant 300 may be used to correct spondylolisthesis. Certain embodiments of the adjustable spinal implant 300 may be used to stabilize the spine during fusion, allowing for controlled load sharing, or selectable unloading of the spine. The adjustable spinal implant 300 may be configured in certain embodiments as an adjustable artificial disc, or to adjust vertebral body height. In treatment of early onset scoliosis, the adjustable spinal implant 300 is secured to the spine 280 of a patient, over the scoliotic curve 296, and is lengthened intermittently by the system 500. In order to obtain the desired growth rate of the spine, a specific force may be determined which is most effective for that patient. Or, an overall average force (for example 20 pounds) may be determined to be effective as a force target during lengthenings (distraction procedures). The system 500 allows the operator to determine whether the target force is reached, and can also protect against too large of a force being placed on the spine 280. In FIG. 26, a distance D is shown between the center of the spinal adjustment device 300 and the spine 280 at the apex vertebra 282. This may be, for example, measured from an X-ray image. The target force may be derived from a target “unbending” moment, defined as:

M_{U}=D×F_{T}

where M_{U} is the target unbending moment, D is the distance D, and F_{T} is the target force.

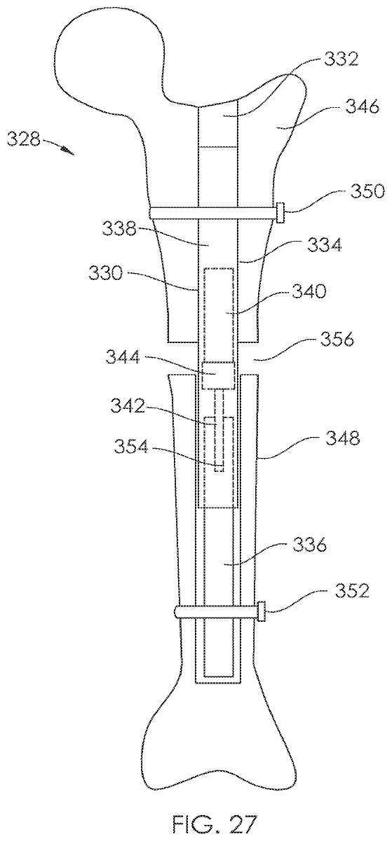

FIG. 27 illustrates a bone 328 with an adjustable intramedullary implant 330 placed within the medullary canal 332. In this particular case, the bone 328 is a femur, though a variety of other bones are contemplated, including, but not limited to the tibia and humerus. The adjustable intramedullary implant 330 includes a first portion 334 having a cavity 338 and a second portion 336, telescopically disposed within the first portion 334. Within the cavity 338 of the first portion 334 is a rotatable permanent magnet 340, which is rotationally coupled to a lead screw 342, first example, via a gear module 344. The first portion 334 is secured to a first section 346 of the bone 328, for example, using a bone screw 350. The second portion 336 is secured to a second section 348 of the bone 328, for example, using a bone screw 352. Rotation of the permanent magnet 340 (for example, by application of an externally applied moving magnetic field of an external adjustment device 700, 502) causes rotation of the lead screw 342 within a female thread 354 that is disposed in the second portion 336, and moves the first portion 334 and the second portion 336 either together or apart. In limb lengthening applications, it may be desired to increase the length of the bone 328, by creating an osteotomy 356, and then gradually distracting the two bone sections 346, 348 away from each other. A rate of approximately one millimeter per day has been shown to be effective in growing the length of the bone, with minimal non-unions or early consolidations. Stretching of the surrounding soft tissue may cause the patient significant pain. By use of the system 500, the patient or physician may determine a relationship between the patient's pain threshold and the force measured by the system 500. In future lengthenings, the force may be measured, and the pain threshold force avoided. In certain applications (e.g., trauma, problematic limb lengthening), it may be desired to place a controlled compression force between the two bone sections 346, 348, in order to form a callus, to induce controlled bone growth, or simply to induce healing, if no limb lengthening is required. System 500 may be used to place a controlled compression on the space between the two bone sections 346, 348.

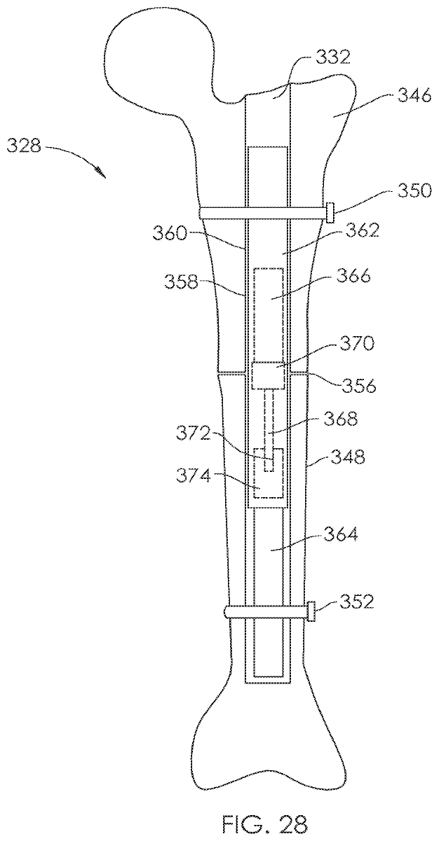

A bone 328 is illustrated in FIG. 28 with an adjustable intramedullary implant 358 placed within the medullary canal 332. In this particular case, the bone 328 is a femur, though a variety of other bones are contemplated, including, but not limited to the tibia and humerus. The adjustable intramedullary implant 358 includes a first portion 360 having a cavity 362 and a second portion 364, rotationally disposed within the first portion 360. Within the cavity 362 of the first portion 360 is a rotatable permanent magnet 366, which is rotationally coupled to a lead screw 368, first example, via a gear module 370. The first portion 360 is secured to a first section 346 of the bone 328, for example, using a bone screw 350. The second portion 364 is secured to a second section 348 of the bone 328, for example, using a bone screw 352. Rotation of the permanent magnet 366 (for example, by application of an externally applied moving magnetic field of an external adjustment device 700, 502) causes rotation of the lead screw 368 within a female thread 372 that is disposed in a rotation module 374, and moves the first portion 360 and the second portion 364 rotationally with respect to each other. The rotation module 374 may make use of embodiments disclosed in U.S. Pat. No. 8,852,187. In bone rotational deformity applications, it may be desired to change the orientation between the first portion 346 and the second portion 348 of the bone 328, by creating an osteotomy 356, and then gradually rotating the bone sections 346, 348 with respect to each other. Stretching of the surrounding soft tissue may cause the patient significant pain. By use of the system 500, the patient or physician may determine a relationship between the patient's pain threshold and the force measured by the system 500. In future rotations, the force may be measured, and the pain threshold force avoided.

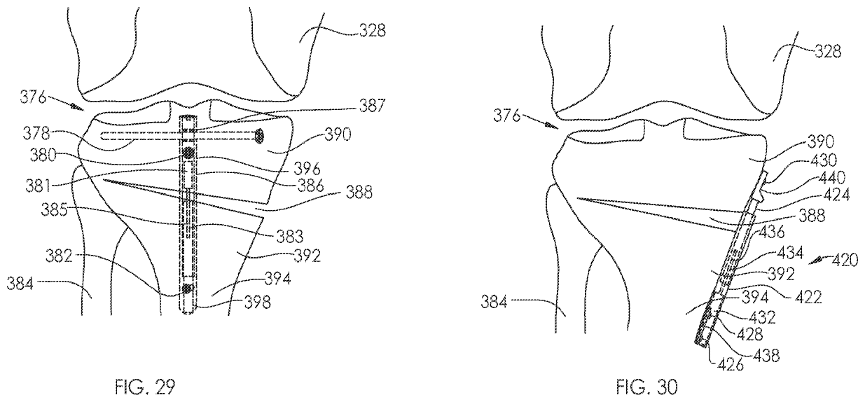

A knee joint 376 is illustrated in FIGS. 29 and 30, and comprises a femur 328, a tibia 394, and a fibula 384. Certain patients having osteoarthritis of the knee joint 376 may be eligible for implants configured to non-invasively adjust the angle of a wedge osteotomy 388 made in the tibia 394, which divides the tibia 394 into a first portion 390 and a second portion 392. Two such implants include an adjustable intramedullary implant 386 (FIG. 29) and an adjustable plate implant 420 (FIG. 30). The adjustable intramedullary implant 386 includes a first portion 396 which is secured to the first portion 390 of the tibia 394 using one or more bone screws 378, 380 and a second portion 398 which is secured to the second portion 392 of the tibia 394 using one or more bone screws 382. A permanent magnet 381 within the adjustable intramedullary implant 386 is rotationally coupled to a lead screw 383, which in turn engages female threads 385 of the second portion 398. In a particular embodiment, the bone screw 378 passes through the adjustable intramedullary implant 386 at a pivoting interface 387. As the angle of the osteotomy 388 is increased with one or more non-invasive adjustments, the bone screw 378 is able to pivot in relation to the adjustable intramedullary implant 386, while still holding the adjustable intramedullary implant 386 securely to the bone of the tibia 394. A rate of between about 0.5 mm and 2.5 mm per day may be effective in growing the angle of the bone, with minimal non-unions or early consolidation. Stretching of the surrounding soft tissue may cause the patient significant pain. By use of the system 500, the patient or physician may determine a relationship between the patient's pain threshold and the force measured by the system 500. In future lengthenings, the force may be measured, and the pain threshold force avoided.

The adjustable plate implant 420 (FIG. 30) includes a first portion 422 having a first plate 438, which is secured externally to the first portion 390 of the tibia 394 using one or more bone screws 426, 428 and a second portion 424 having a second plate 440, which is secured externally to the second portion 392 of the tibia 394 using one or more bone screws 430. A permanent magnet 432 within the adjustable plate implant 420 is rotationally coupled to a lead screw 434, which in turn engages female threads 436 of the second portion 424. Stretching of the surrounding soft tissue may cause the patient significant pain. By use of the system 500, the patient or physician determine a relationship between the patient's pain threshold and the force measured by the system 500. In future lengthenings, the force may be measured, and the pain threshold force avoided.

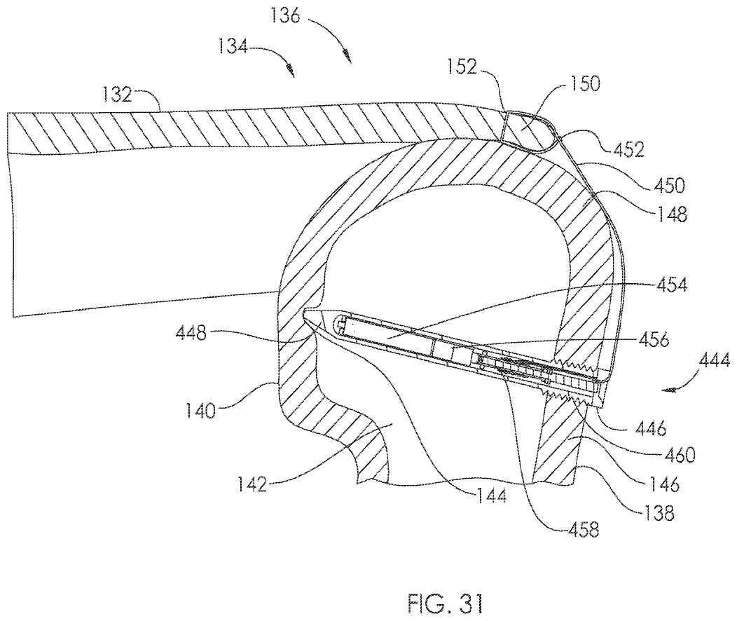

An adjustable suture anchor 444 is illustrated in FIG. 31. Though the embodiment is shown in a rotator cuff 134 of a shoulder joint 136, the adjustable suture anchor 444 also has application in anterior cruciate ligament (ACL) repair, or any other soft tissue to bone attachment in which securement tension is an factor. The adjustable suture anchor 444 comprises a first end 446 and a second end 448 that is configured to insert into the head 140 of a humerus 138 through cortical bone 146 and cancellous bone 142. Threads 460 at the first end 446 are secured to the cortical bone 146 and the second end 448 may additionally be inserted into a pocket 144 for further stabilization. Suture 450 is wound around a spool 458 within the adjustable suture anchor 444, extends out of the adjustable suture anchor 444, and is attached to a tendon 150 of a muscle 132 through a puncture 152 by one or more knots 452, for example, at the greater tubercle 148 of the humerus 138. A permanent magnet 454 is rotatably held within the adjustable suture anchor 444 and is rotatably coupled to the spool 458, for example via a gear module 456. It may be desirable during and/or after surgery, to keep a muscle secured to a bone at a very specific range of tensions, so that healing is maximized and range of motion is optimized. Using the system 500, the force may be measured, adjusted accordingly, at surgery, immediately after surgery, and during the healing period in the weeks after surgery).

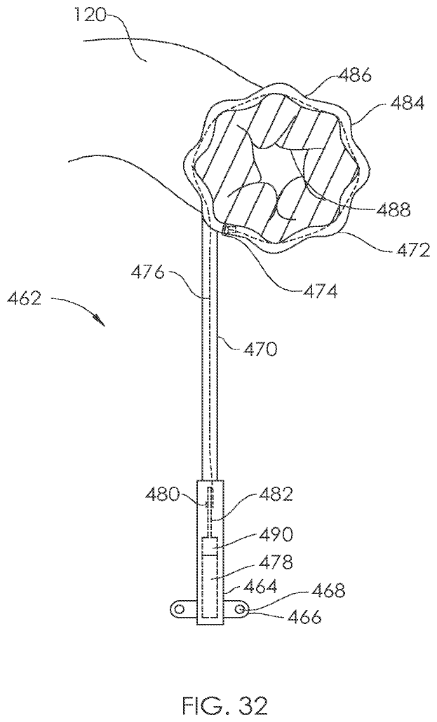

FIG. 32 illustrates an adjustable restriction device 462 having an adjustable ring 472 which is configured to be secured around a body duct 120 and closed with a closure or snap 474. The adjustable restriction device 462 may be implanted in a laparoscopic surgery. A housing 464 having suture tabs 466 is secured to the patient, for example, by suturing though holes 468 in the suture tabs 466 to the patient's tissue, such as fascia of abdominal muscle. Within the housing 464 is a magnet 478 which is rotationally coupled to a lead screw 482. A nut 480 threadingly engages with the lead screw 482 and is also engaged with a tensile line 476, which may comprise wire, for example Nitinol wire. The tensile line 476 passes through a protective sheath 470 and passes around the interior of a flexible jacket 484 that makes up the adjustable ring 472. The flexible jacket 484 may be constructed of silicone, and may have a wavy shape 486, that aids in its ability to constrict to a smaller diameter. The duct 120 is shown in cross-section at the edge of the adjustable ring 472, in order to show the restricted interior 488 of the duct 120. Certain gastrointestinal ducts including the stomach, esophagus, and small intestine may be adjustably restricted. Sphincters such as the anal and urethral sphincters may also be adjustably restricted. Blood vessels such as the pulmonary artery may also be adjustably restricted. During adjustment of the adjustable restriction device 462, an external adjustment device 700, 502 is placed in proximity to the patient and the magnet 478 is non-invasively rotated. The rotation of the magnet 478 rotates the lead screw 482, which, depending on the direction of rotation, either pulls the nut 480 toward the magnet 478 or pushes the nut away from the magnet 478, thereby either increasing restriction or releasing restriction, respectively. Because restricted ducts may have complex geometries, their effective size is hard to characterize, even using three-dimensional imaging modalities, such as CT or MRI. The force of constriction on the duct may be a more accurate way of estimating the effective restriction. For example, a stomach is restricted with a tangential force (akin to the tension on the tensile line 476) on the order of one pound. With a fine lead screw having about 80 threads per inch, a fine adjustment of the nut 480, and thus of the adjustable ring may be made. By including a gear module 490 between the magnet 478 and the lead screw 482, and even more precise adjustment may be made. By use of the system 500, the force may be measured, during adjustment, so that an “ideal restriction” may be returned to after changes occur in the patient (tissue growth, deformation, etc.).

FIG. 34 illustrates an external adjustment device 1100 having one or more magnets 1106, 1108 which may comprise permanent magnets or electromagnets, as described in other embodiments herein. In some applications, one or more of the Hall effect sensors 534, 538, 540 may experience an undesired amount of saturation. An upper leg portion 1102 having a bone 1118 extending within muscle/fat 1116 and skin 1104 is shown in FIG. 34. An implant 1110, such as a limb lengthening implant, having a magnet 1010 is placed within the medullary canal of the bone 1118. In large upper leg portions 1102, for example in patients having a large amount of muscle or fat 1116, the distance “A” between the magnet 1010 and the Hall effect sensors 534, 538, 540 decreases the signal the magnet 1010 can impart on the Hall effect sensors 534, 538, 540 thus increasing the relative effect the one or more magnets 1106, 1108 have on the Hall effect sensors 534, 538, 540. The external adjustment device 1100 includes one or more Hall effect sensors 597, 599 spaced from the one or more magnets 1106, 1108. The one or more Hall effect sensors 597, 599 may be electrically coupled to the external adjustment device 1100 directly or remotely. In some embodiments, the one or more Hall effect sensors 597,599 may be mechanically attached to the external adjustment device 1100, or may be attachable to the body of the patient, for example to the upper leg portion 1102. Distances B and C may each range between about 5 cm and 15 cm, between about 7 cm and 11 cm, or between about 8 cm and 10 cm. In some embodiments, one or both of the Hall effect sensors 597, 599 may include a shield 1112, 1114, such as a plate. The shield may comprise iron or MuMETAL®, (Magnetic Shield Corporation, Bensenville, Ill., USA). The shield may be shaped or oriented in a manner such that it is not between the particular Hall effect sensor 597, 599 and the magnet 1010, but is between the particular Hall effect sensor 597, 599 and the one or more magnets 1106, 1108. The Hall effect sensors 597, 599 may each be used to acquire a differential voltage, as described in relation to the other Hall effect sensors 534, 538, 540. Larger distances between that the Hall effect sensors 597, 599 and the one or more magnets 1106, 1108 can advantageously minimize the amount of saturation due to the magnets 1106, 1108. Additionally, the shield 1112, 1114 can significantly minimize the amount of saturation.

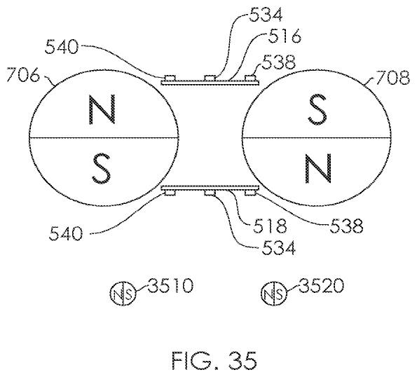

FIG. 35 is a front view of an arrangement of magnetic sensors (e.g., Hall effect sensors (“HES”) in an embodiment of an external adjustment device that may be used with two adjustable implants 3510, 3520. The external adjustment device shown is similar to that shown in FIG. 17. For example, there is a first external magnet 706 and a second external magnet 708. In differential mode, the left HES 538 of circuit board 516 is paired with the right HES 540 of circuit board 518. The left HES 538 of circuit board 518 is paired with the right HES 540 of circuit board 516. Any center HES 534, 542, 536 of circuit board 516 is paired with the corresponding center HES 534, 542, 536 of circuit board 518. Thus, in differential mode there may be at least three pairs of hall-effect sensors.

Unlike the system shown in FIG. 17, here there are two adjustable implants: a left adjustable implant 3510 and a right adjustable implant 3520. All three pairs of hall-effect sensors are picking up the magnetic fields of left adjustable implant 3510 and right adjustable implant 3520. In differential mode, the top sensors 540, 534, 538 of circuit board 516 may have little pickup/detection of the internal magnets in left adjustable implant 3510 and right adjustable implant 3520, but instead pickup/detect the first external magnet 706 and second external magnet 708. The bottom sensors 540, 534, 538 of circuit board 518 have pickup/detection of the left adjustable implant 3510 and the right adjustable implant 3520 as well as the pickup/detection of first external magnet 706 and second external magnet 708. Thus, for each pair of HES, the measurement of the top sensor in the pair is subtracted from the measurement of the bottom sensor in the pair, which can be the form of a voltage differential. The pickup/detection of the first external magnet 706 and second external magnet 708 may be subtracted, leaving just the pickup/detection of the left adjustable implant 3510 and right adjustable implant 3520.

The external adjustment device may be placed along the midline between the left adjustable implant 3510 and right adjustable implant 3520. In one configuration, the external adjustment device can be used to distract both implants at the same time. For example, the implants may be placed bilaterally on each side of the spine of a patient. The left adjustable implant 3510 could be on the left side of the spine and the right adjustable implant 3520 could be on the right side of the spine. To adjust the implants, the external adjustment device could be placed over the spine (e.g., over or above both implants). Using the external adjustment device may cause bilateral actuation (e.g., distraction of both implants, retraction of both implants, or retraction of one implant and distraction of the other implant) allowing for the generation of more force on the spine of the patient. In another configuration (not shown), the external adjustment device can be used to simultaneously distract one implant while retracting another implant. In the example of having an implant on each side of the spine of a patient, this configuration would end up bending the spine in the direction of the implant that is being retracted and may be beneficial in correcting curvatures in the spine of a patient. The implants may also be adjusted so that each imparts a different force on the spine. For example the left implant 3510 may be adjusted so that it imparts a larger force on the spine than the right implant 3520. Or, the right implant 3520 may be adjusted so that it imparts a larger force on the spine than the left implant 3510.

Using only the middle pair of HES, coupling between the external adjustment device 700 and both implants, centering between the two implants, and offsets can be determined. With just the middle sensor pair, centering can be performed by making sure the pickups of the magnetic field from the left implant 3510 and the right implant 3520 are substantially equivalent. The middle sensor pair can also be used for offset, so that the external adjustment device 700 can be placed directly over either the left implant 3510 or the right implant 3520. The middle sensor pair can also be used for coupling detection between the external adjustment device 700 and one or both implants. For example, the device can be considered coupled if the middle sensor pair is picking up/registering a voltage differential above a coupling threshold value that takes into account the magnetic fields from both the left implant 3510 and the right implant 3520.

However, using the middle pair of HES alone it may be difficult to determine which implant of the two is stalling or slipping. When the external adjustment device 700 is properly positioned in the middle of the two implants, the middle pair of HES would likely be substantially equally affected by both the left implant 3510 and the right implant 3520, and thereby unable to monitor either implant independently. Three pairs of HES, as shown in the figure, may advantageously allow the determination of which implant is stalling. The sensor pair of bottom sensor 540 of circuit board 518 and top sensor 538 of circuit board 516 (the left pair) would be closest to the left implant 3510 and be affected mainly by the left implant 3510 (although it may pick up some of the magnetic field of right implant 3520). The sensor pair of bottom sensor 538 of circuit board 518 and top sensor 540 of circuit board 516 (the right pair) would be closest to the right implant 3520 and be affected mainly by the right implant 3520 (although it may pick up some of the magnetic field of left implant 3510). The voltage differentials of each of these side pairs may be monitored to determine slippage of its respective implant according to substantially the same process outlined above for determining slippage of one implant. Each side pair of sensors may also be used to monitor coupling with its respective implant based on whether the voltage differential is above or greater than a coupling threshold value. Alternatively the left pair may comprise bottom sensor 540 of circuit board 518 and the right pair may comprise top sensor 540 of circuit board 516. Or, the left pair may comprise bottom sensor 538 of circuit board 518 and the right pair may comprise top sensor 538 of circuit board 516.

The amplitude of the voltage differential for each sensor pair is associated with the amount of force generated by the implants (e.g., amplitude may increase if resistance force on the implants or force generated by the implants increases). Based on this relationship, the force on the implants can be measured through the amplitude of the voltage generated. For the left sensor pair, the measured amplitude may be dominated by the force on/force generated by the left implant 3510. For the right sensor pair, the measured amplitude may be dominated by the force on/force generated by the right implant 3520. However, other scenarios may be observed. For example, the measured amplitude for the left sensor pair could be affected more by the right implant if the left implant is experiencing/generating little force, but the right implant is experiencing/generating a significantly higher amount of force. In that case, the right implant 3520 would be disproportionately influencing the measured amplitude considering it is farther in distance from the left sensor pair than is the left implant 3510.

In some embodiments, the amplitudes of the voltage measured by the left and right sensor pairs may be compared in order to determine the portion of the amplitude that is attributable to each sensor pair's respective implant. For example, comparing the left sensor pair amplitude and the right sensor pair amplitude could allow the influence of the right implant 3520 to be subtracted out of the left sensor pair amplitude, allowing a determination of the force being exerted on just the left implant 3510, and vice versa. Furthermore, the sensor pairs and the two implants may be configured to better allow for measurement of the force exerted on each implant. For example, the spacing between the two implants could be far enough apart, or the strength of the magnets of each implant adjusted, such that the amplitude measured by the right sensor pair is not influenced by the left implant 3510. Additional sensor pairs may also be added to the external adjustment device in order to accommodate additional simultaneous implants, ensuring that each additional sensor pairs would be located as close as possible to the particular implant it is tasked with monitoring.

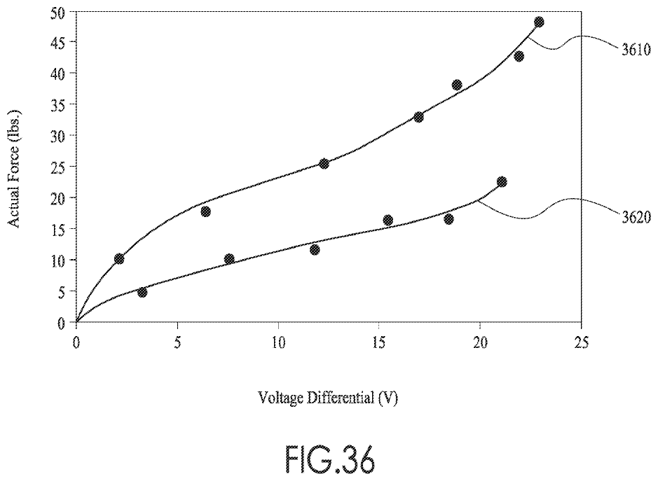

FIG. 36 is a graph of actual force against voltage differential for two different gap distances. Curve 3610 may be a curve-fit model for the force-voltage relationship for a gap distance of 10 mm. Curve 3620 may be a curve-fit model for the force-voltage relationship for a gap distance of 20 mm. These models may be used to predict how much force is being applied to/generated by an implant for a particular gap distance and measured voltage amplitude. For example, different forces and voltages can be measured at a gap distance of 10 mm, 20 mm, and so forth. The data can be compiled into a lookup table and/or used for curve-fitting to generate the model. As a non-limiting example of a measured range of gap distances, force and voltage data for a gap distance between 6-25 mm at an interval of 5 mm may be measured. Thus, enough data could be gathered in order to curve-fit the models for curve 3610 and curve 3620 in the figure.

A user of the external adjustment device may enter an estimated gap distance into the user interface of the external adjustment device, such as through the data input module 248 shown in the user interface of FIG. 21. This estimated gap distance may be measured by the user in a variety of ways, such as by taking a medical imaging scan of the patient to determine the distance from an implant to the surface of the patient's skin at which the external adjustment device would be positioned, including, but not limited to ultrasound, X-ray, or computed tomography (CT). The estimated gap distance can be used to determine the proper curve-fit model to use in order to estimate force for a given voltage amplitude. For an estimated gap distance without a corresponding curve-fit model, the calculation could be performed by interpolating between two existing curve-fit models. For instance, if no curve-fit model were available for gap distances between 10 mm and 20 mm and a patient had an estimated gap distance of 15 mm, then the calculation could be accomplished by interpolating between curve 3610 and curve 3620. The interpolation need not be linear, and may include powered or other non-linear interpolation based on, for example, inverse-square, inverse-cube, or other relationships.

In some embodiments, the external adjustment device may be able to directly estimate the gap distance. For example, the external adjustment device may have coils. The coils may be configured not to saturate, unlike hall-effect sensors. The coils may be used to detect and measure the flux through the coils created by the magnetic fields of the implants along with the magnets of the external adjustment device. However, the coils may be fixed with respect to the external adjustment device (e.g., fixed in or on the housing of the external adjustment device) thereby rendering any measured flux from magnets in the external adjustment device also fixed (e.g., a wave that can simply be processed out). Thereby any changes to the flux in the coils may be due to the magnetic fields of the implants. As an implant moves closer to each coil, more flux in the coil may result. Conversely, as an implant moves away from each coil, less flux in the coil may result. Thus, the flux in the coil can be used to determine the distance between the coil and the implant, which, in turn, may then be used to determine the gap distance. Furthermore, coils may also be used to determine coupling, slippage of the implant, and so forth. More information about the implementation of coils is provided in the discussion with regard to FIGS. 42-45

FIGS. 37A and 37B illustrate embodiments of an adjustable implant configured to reduce the number of instances in which the measured or estimated distraction length could be different from (e.g., considerably different from) the actual distraction length. For example, if a coupler (e.g., a coupling pin) between the internal permanent magnet and the lead screw were broken, a user may erroneously assume that the lead screw is being rotated and the adjustable implant is being distracted. More information regarding this is provided above in association with FIG. 20.

FIG. 37A illustrates an embodiment of an adjustable implant in which the lead screw incorporates a magnet. This adjustable implant may be similar to the other adjustable implants or distraction devices 1000 discussed above. There is a first portion 3701 and a second portion 3702, which may also be referred to as a distraction rod. An internal permanent magnet 3703 is configured to be coupled to an external adjustment device and be magnetically rotated. The magnet 3703 is mechanically coupled to a lead screw 3705 via a gear box 3704 (which may be optional), which may include a coupling pin. The lead screw 3705 is configured to mate with a threaded recess (e.g., a nut) in distraction rod 3702. As the magnet 3703 is rotated, the lead screw 3705 is rotated so that, through interaction with the nut, it causes the entire length of the adjustable implant to be distracted or retracted. This embodiment may also incorporate a magnet 3706 in or as part of the lead screw 3705. It should be noted that magnet 3706 is shown at the tip of the lead screw 3705, but it can be located anywhere on or within the lead screw. The rotation of magnet 3706 can be measured by the external adjustment device (e.g., the Hall effect sensors discussed above, or additional Hall effect sensors included in other parts of the external adjustment device) in order to directly determine the rotation of the lead screw 3705. Thus, a user can determine how much the lead screw 3705 is rotating even if the coupling pin in gear box 3704 is broken, allowing for more reliable calculations of the distraction length of the adjustable implant using the methods disclosed herein.

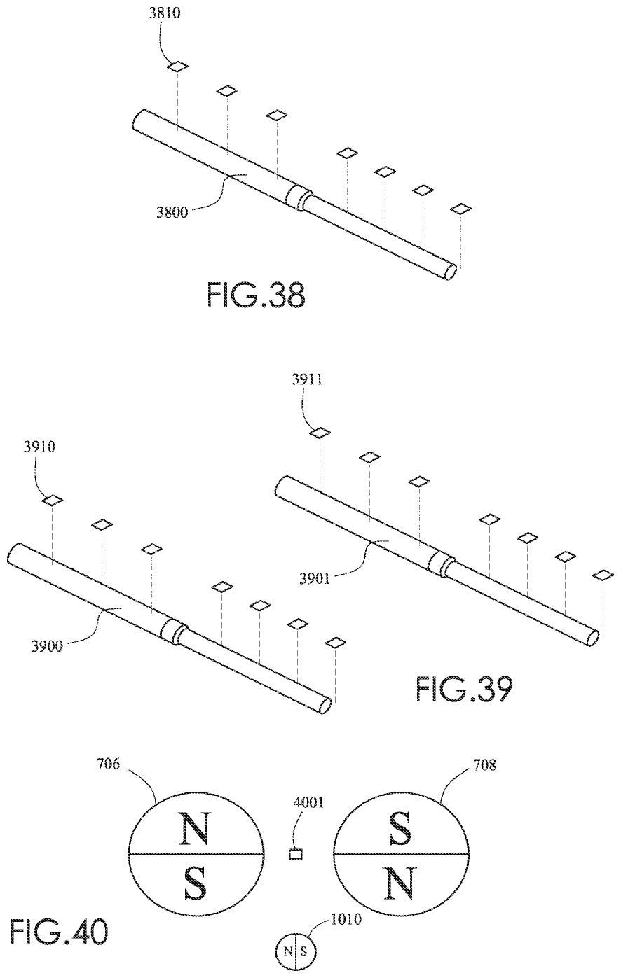

FIG. 37B illustrates an embodiment of an adjustable implant in which the distraction rod has or incorporates a magnet. This adjustable implant may also be similar to the other adjustable implants or distraction devices 1000 discussed above. There is a first portion 3711 and a second portion 3712, which may also be referred to as a distraction rod. An internal permanent magnet 3713 is configured to be coupled to an external adjustment device and be magnetically rotated. The magnet 3713 is mechanically coupled to a lead screw 3715 via a gear box 3714 (which may be optional), which may include a coupling pin. The lead screw 3715 is configured to mate with a threaded recess (e.g., a nut) in distraction rod 3712. As the magnet 3713 is rotated, the lead screw 3715 is rotated so that, through interaction with the nut, it causes the entire length of the adjustable implant to be distracted or retracted. This embodiment may also incorporate a magnet 3716 in or as part of the distraction rod or second portion 3712. The distance between the internal permanent magnet 3713 and the magnet 3716 can be used to determine the actual distraction length. The distraction rod moves linearly, whereas the lead screw rotates. By having magnet 3716 on the distraction rod as it distracts or retracts linearly, the absolute distance between the two magnets may be determined rather than having to indirectly calculate distraction distance by counting revolutions of the lead screw. It should be noted that magnet 3716 can be located at or within any position within the second portion 3712. The dimensions of every portion of the adjustable implant may be known before-hand, so even if magnet 3716 is not placed at the tip of the distraction rod, the total distraction length can be determined based on how far the magnet 3716 is from the tip. Methods for determining the distance between the two magnets are described below in association with FIGS. 38 and 39.

FIG. 38 illustrates an array of magnet sensors for use with an embodiment of an adjustable implant in which the distraction rod has a magnet. The adjustable implant 3800 may be the same as the adjustable implant of FIG. 37A, which has two magnets: a first magnet housed in the first portion and a second magnet housed in the second portion (the distraction rod). An array of hall-effect sensors 3810 is arranged external to the patient and axially (e.g., positioned along an axis parallel to the longitudinal axis of the implant magnet) to the adjustable implant 3800, with the individual hall-effect sensors spaced at approximately equal, pre-determined distances. The array of hall-effect sensors 3810 may then be used to determine the distance between the two magnets. The array of hall-effect sensors 3810 may alternatively be an array of hall-effect sensor pairs.

A simplified example is that the hall-effect sensor located near the first magnet will receive a strong signal associated with the first magnet. The hall-effect sensor that is located near the second magnet will receive a strong signal associated with the second magnet. While the other hall-effect sensors in the array may receive a signal associated with either magnet, the signals they pick up will not be as strong. A known threshold value can be used for determining whether each magnet is directly positioned under a hall-effect sensor. After determining which two hall-effect sensors are nearest to each of the first and second magnets, the distance between the magnets may be determined based on the known distance between each hall-effect sensor.

Practically however, the process may be more complex. The magnets may be positioned between hall sensors. An algorithm may be used to determine the position of the magnets when they are located between hall-effect sensors by comparing the signal values of the surrounding hall-effect sensors. For example, if a magnet is directly between two hall-effect sensors, then those two hall-effect sensors should have signal values that represent a local peak in comparison to the surrounding hall-effect sensors. Thus, a peak-detection algorithm may be used to determine the two hall-effect sensors closest to the position of each magnet. The signal values at those two hall-effect sensors can then be compared to determine positioning of the magnet. For example, if the magnet is directly between two hall-effect sensors, their signal values should be very close, if not substantially equal. If the magnet is slightly closer to one hall-effect sensor, the signal value at that hall-effect sensor should be greater. Increasing the number of hall-effect sensors in the array of hall-effect sensors 3810 may increase the resolution at which the position of each magnet may be determined. After the positions of the two magnets are determined, then the distance between them may be calculated based on known information. That distance can be used to determine the entire distraction length based on the known dimensions of the adjustable implant 3800.

In some embodiments, the array of sensors 3810 is a separate device that may be electronically tethered to the main external adjustment device. The location of the first magnet is known since it is coupled to the external adjustment device (e.g., magnetically coupled), and that is taken to be the reference location. The tethered sensors may then be used to determine the position of the second magnet in the distraction rod. For this approach, the distance between the external adjustment device and the tethered sensors would have to be measured and compensated for. In some embodiments, the tethered sensors may be placed on or fixed onto the patient.

In some embodiments, the array of sensors 3810 is actually an array within the external adjustment device itself. For example, FIG. 18 illustrates an external adjustment device with an array of sensor pairs 534, 542, 536 positioned relative to the adjustable implant 1010. More specifically, the forward HES 534 (or any other center HES, e.g., 536 or 542) of circuit board 516 is paired with the forward HES 534 (or any other center HES, e.g., 536 or 542) of circuit board 518. The middle HES 542 of circuit board 516 is paired with the middle HES 542 of circuit board 518. And, the back HES 536 of circuit board 516 is paired with the back HES 536 of circuit board 518. In one embodiment, the array of sensors 3810 is configured to measure distraction length up to 3-4 inches depending on the strength of the second magnet embedded in the distraction rod.

In some embodiments, the array of sensors 3810 may be used to determine the distraction length of two implants. The singular array may be appropriate when the two implants are distracted to different lengths such that the signal value peaks from each implant are distinct and identifiable. However, if the distraction lengths of the two implants are close together, then a single array of sensors may be unable to distinguish between the two implants. In that case, two arrays of sensors may be used as shown in FIG. 39.

FIG. 39 illustrates using multiple arrays of magnetic sensors for use with embodiments of an adjustable implant in which the distraction rod has a magnet. Both adjustable implant 3900 and adjustable implant 3901 may be similar to the adjustable implant in FIG. 37B, in that they have two magnets each, with a magnet within the distraction rod. A first array of hall-effect sensors 3910 is used for measuring distraction length of adjustable implant 3900, and a second array of hall-effect sensors 3911 is used for measuring distraction length of adjustable implant 3901. This setup can be generalized for more implants, such that N arrays of hall-effect sensors are used with N implants, with an array of sensors measuring the distraction length for each implant.

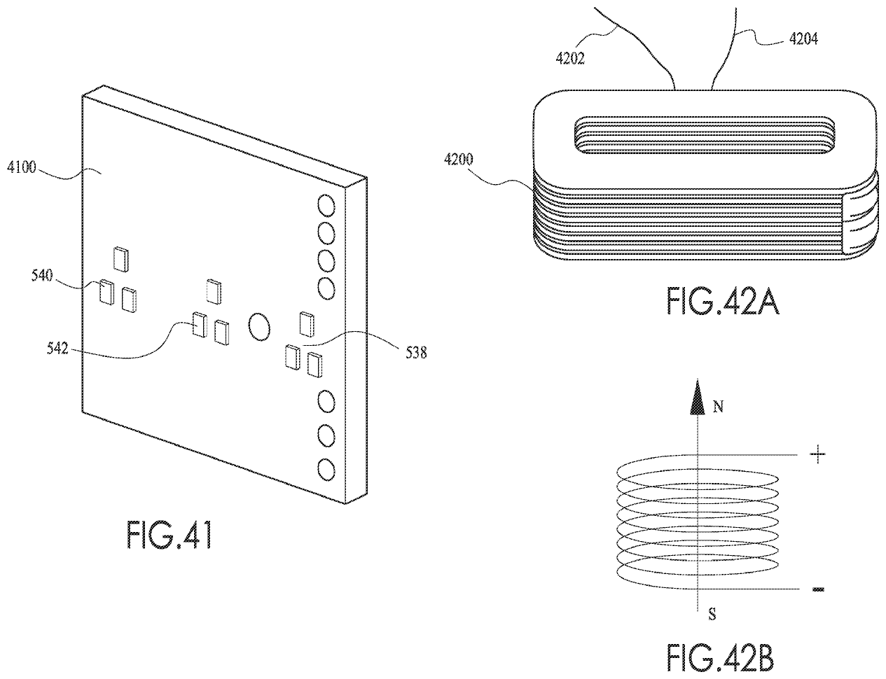

FIG. 40 is a front view of a magnetic sensor in an embodiment of an external adjustment device. This figure is similar to FIG. 17, however this external adjustment device has a single magnetic sensor 4001, such as a hall-effect sensor. Magnetic sensor 4001 is positioned at the midpoint of first external magnet 706 and second external magnet 708, such that the measured/experienced flux from both magnets is zero (e.g., cancels each other out). The positioning of magnetic sensor 4001 allows it to ignore the effects of (or not register the effects of) magnets 706 and 708 while still being able to detect the magnetic field of an adjustable implant or distraction device in order to detect coupling or stalling. A differential mode of operation is therefore not needed, thereby reducing the number of hall-effect sensors used, since the flux from the magnets 706 and 708 cancel each other out. Instead of a single sensor, an array of sensors (e.g., an array of sensors axial to the adjustable implant) could be positioned within this zone

FIG. 41 is a perspective view of an arrangement of magnetic sensors on circuit board 4100 in an embodiment of an external adjustment device. This arrangement is similar to circuit board 516 shown in FIG. 15. Circuit board 4100 also has Hall effect sensors 538, 540, and 542 in the same linear arrangement of circuit board 516. However, the circuit board 4100 shown only has those three Hall effect sensors and does not have Hall effect sensors 534 and 536 on circuit board 516. Thus, the Hall effect sensors 538, 540, and 542 in circuit board 4100 may have a matching or corresponding circuit board on the opposing side of the external magnets, and that corresponding board may have a similar arrangement of Hall effect sensors as those shown on circuit board 4100.

The three pairs of Hall effect sensors between the two circuit boards may be used to indirectly measure the status of one or more implants. In the case of two implants, the three pairs of Hall effect sensors may be used as described in regards to FIG. 35. In the case of one implant, the three pairs of Hall effect sensors may all be used to monitor the singular implant and provide redundancy in measurements. The two pairs of Hall effect sensors to the sides of the implant may be used to confirm measurements of the central pair of Hall effect sensors that includes both sensors 542 that reside on circuit board 4100 and its corresponding circuit board.

However, in some embodiments of the external adjustment device, only one pair of Hall effect sensors may actually be used on a single implant. This may be any one of the pairs of Hall effect sensors. It may be the central pair of Hall effect sensors, which would include Hall effect sensors 542 that reside on circuit board 4100 and its corresponding circuit board. In some embodiments, the other Hall effect sensors 538 and 540 are not used. A differential voltage between the Hall effect sensor 542 on circuit board 4100 and the Hall effect sensor 542 of the opposing circuit board may be analyzed to determine whether the external adjustment device and the implant are sufficiently coupled such that they are both oriented correctly and positioned closely enough to each other for a sufficient magnetic interaction between the two. The differential voltage may also be analyzed to determine whether there is slippage or stall between the magnets of the external adjustment device and the implant. The differential voltage may also be analyzed to determine the degree or amount of coupling strength between the magnets of the external adjustment device and the implant.

FIG. 42A illustrates a wire coil 4200 for use with an embodiment of an external adjustment device. This wire coil may be referred to as an inductive coil, induction magnetometer, search coil, and/or search coil magnetometer. Wire coil 4200 may be a loop of wire with the ends of the wire connected to a circuit in order to supply a current in the wire. It can operate as sensor to measure the variation of magnetic flux, and it can be configured to have a sensitivity tailored to a specific purpose. It may measure magnetic fields ranging from mHz up to hundreds of MHZ.

The wire coil 4200 operates based on Faraday's law. Any change or variation of magnetic flux through the wire coil 4200 will induce a change in voltage in the circuit. For example, the change in magnetic flux may result from changing the magnetic field strength, moving a magnet toward or away from wire coil 4200, moving the wire coil 4200 into or out of the magnetic field, and/or rotating the wire coil 4200 relative to the magnet. The change in voltage that is induced in the circuit is proportional to the number of turns in the wire coil 4200. Since the number of turns can be known in advance and in practice would be pre-defined by the wire coil 4200 manufacturer, the relationship between the change in magnetic flux through wire coil 4200 and the measured change in voltage of the circuit may be well defined. Wire coil 4200 may be fixed in position and orientation on the external adjustment device, such that the magnetic flux from the external magnets of the external adjustment device is constant and/or known. Any measured variations in magnetic flux through wire coil 4200 could then be a result of magnetic fields external to the external adjustment device.

The wire coil may also be wound around a ferromagnetic or similarly magnetic core, which increases the sensitivity of the sensor due to the apparent permeability of the ferromagnetic core. This arrangement may be an electromagnet, in which the strength of the magnetic field generated is proportional to the amount of current travelling through the winding. Wire coil 4200 may be in any shape, and not necessarily circular coils. For example it could be wound in a way that it has a substantially rectangular cross-section. In some embodiments, a wire coil may have rectangular dimensions of 1″×¼″, and the corners may be slightly curved as shown in FIG. 42A.

FIG. 42B illustrates a schematic representation of a wire coil (e.g., the wire coil 4200 illustrated and discussed in regard to FIG. 42A). In the figure, a coil of wire can be seen with ends connected to a circuit to yield a positive end and a negative end. A magnetic field line is shown passing through the center of the wire coil. Variations in the magnetic flux through the center of the wire coil correspond to a change in the voltage of the circuit.

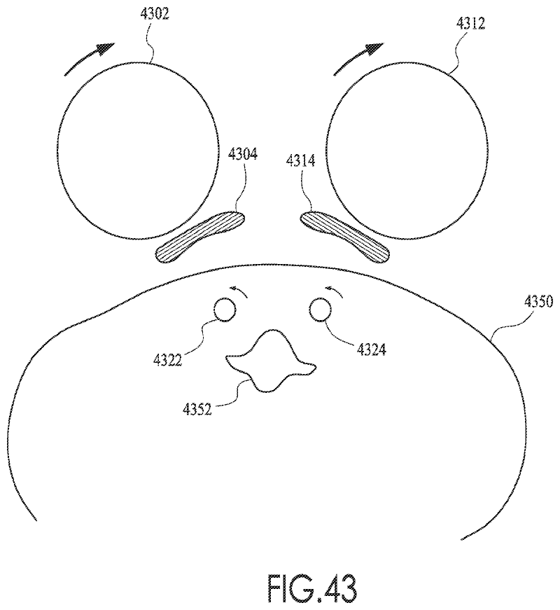

FIG. 43 illustrates an embodiment of an external adjustment device having two wire coils being used on two adjustable implants in a patient. In this embodiment, there is a first coil 4304 and a second coil 4314 within the external adjustment device. The two coils are fixed in position and orientation such that the magnetic flux passing through them due to first external magnet 4302 and second external magnet 4312 remains constant.

Patient 4350 has a first adjustable implant 4322 and a second adjustable implant 4324 implanted within them. In this example, the two implants are on either side of vertebra 4352 of patient 4350, which may be an implant arrangement for treating a spinal disorder of patient 4350 such as scoliosis. Once the external adjustment device is appropriately coupled to the two adjustable implants, the two adjustable implants may be retracted and/or distracted at the same time. In the figure, the first external magnet 4302 and second external magnet 4312 are shown to be rotating clockwise. This creates a magnetic force that acts on first adjustable implant 4322 and second adjustable implant 4324 and spins an internal magnet within each implant counterclockwise. This may result in retraction or distraction of each implant, depending on the orientation of each implant and the orientation of the threading between the lead screw and the distraction rod of each implant.

First coil 4304 and second coil 4314 may take on all or some of the functions for which Hall effect sensors may be used as described in other example embodiments. In some embodiments, first coil 4304 and second coil 4314 completely replace the use of Hall effect sensors (e.g., there are no Hall effect sensors at all in the external adjustment device). In some embodiments, first coil 4304 and second coil 4314 may have complementary, or even overlapping, functions with any Hall effect sensors included in the external adjustment device. For example, the Hall effect sensors may be used to measure coupling, slippage, and force while the coils may be used to determine the gap distance between the external adjustment device and the implants. In some embodiments, first coil 4304 and second coil 4314 may have the same functions as Hall effect sensors and either the coils or Hall effect sensors may be used for redundancy or measurement-checking. For example, the coils and Hall effect sensors may all be used to measure coupling, slippage, and force. The coils could be confirming the measurements of the Hall effect sensors, or vice versa, such that if there is too much of a deviation between the measurements of the two kinds of sensors the external adjustment device may turn off or trigger an alarm/update to the user. It should be noted that in all of these described embodiments, there may be any number of coils and/or Hall effect sensors.

In one embodiment, the first coil 4304 and second coil 4314 replace all the Hall effect sensors and are configured for use with two implants. The first coil 4304 is positioned and oriented in order to measure variations of magnetic flux primarily due to the first implant 4322 and second coil 4314 is positioned and oriented in order to measure variations of magnetic flux primarily due to the second implant 4324. FIG. 43 illustrates how placing the external adjustment device over the implants results in the first coil 4304 being in proximity to first implant 4322 and second coil 4314 being in proximity to second implant 4324. Although first coil 4304 may measure some of the magnetic flux coming from the second implant 4324 and second coil 4314 may measure some of the magnetic flux coming from the first implant 4322, the variations in magnetic flux measured by the coils may be predominantly due to the magnetic flux of the implant to which each coil is closest.

Additionally, the two coils may be used to estimate gap distance. As the coils approach the implants, the magnetic flux through the coils will increase. Such information can be used to estimate the distance between each coil and the closest implant. This information may also be used to detect coupling between the external adjustment device and the implants. The two coils may also be used to sense slippage of any internal magnets within the implants.

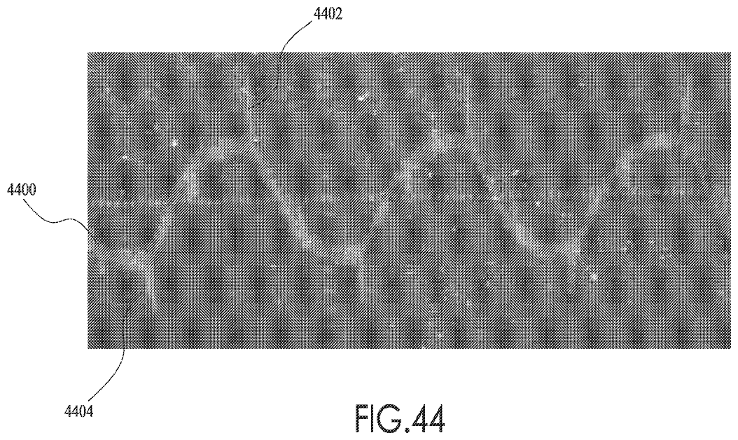

FIG. 44 illustrates a graph of a signal 4400 generated based on magnetic flux through a wire coil of an embodiment of the external adjustment device. This information can be used to determine which of the two implants, if either, is experiencing slippage when both implants are being distracted by the external adjustment device. Spikes 4402 and 4404 in the signal 4400 represent the occurrence of slippage. However, it may be difficult to determine from this image which implant is experiencing slippage. It could be slippage from the implant closest in proximity to that wire coil, such as slippage from first implant 4322 if the wire coil was first coil 4304. However, it could also be slippage occurring in the farther implant (assuming a strong signal is being registered by the coil), such as slippage from second implant 4324 being picked up by first coil 4304. Readings from two coils can be used to determine which implant is experiencing slippage.

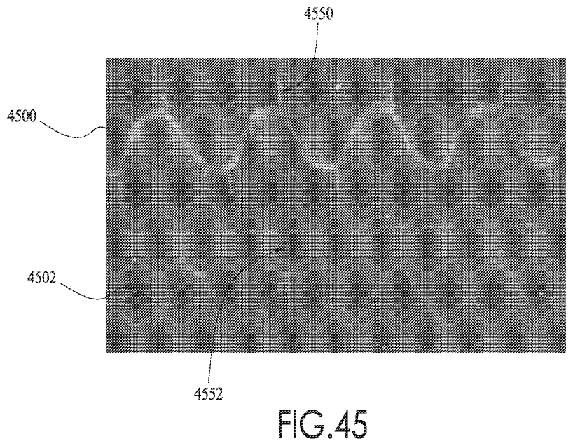

FIG. 45 illustrates graphs of signals generated based on magnetic flux through two wire coils of an embodiment of the external adjustment device. Signal 4500 is generated from one coil and signal 4502 is generated from the other coil. The timelines of both signal 4500 and signal 4502 are aligned for comparison. Signal 4502 shows one coil detecting a slippage spike 4552 between an external and internal magnet, when at the same time signal 4500 has a much weaker slippage spike 4550. This means that the implant closest to the coil that generated signal 4502 is slipping, while the other implant (which is closer to the coil generating signal 4500) is likely not slipping at that particular moment.

It is understood that any specific order or hierarchy of steps in any disclosed process is an example of a sample approach. Based upon design preferences, it is understood that the specific order or hierarchy of steps in the processes may be rearranged while remaining within the scope of the present disclosure. The accompanying method claims present elements of the various steps in a sample order, and are not meant to be limited to the specific order or hierarchy presented.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles, and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. A medical implant configured for magnetic adjustment within a body of a patient, the medical implant comprising:
a rod configured to be coupled to a first location within the body of the patient;
a housing configured to be coupled to a second location within the body of the patient and having a first magnet disposed therein, the first magnet configured to revolve around an axis, wherein the housing is configured to telescopically receive the rod; and
a lead screw having a first end and a second end opposing the first end, the second end of the lead screw being fixed relative to the first end of the lead screw, wherein the first end of the lead screw is mechanically coupled to the first magnet and the second end of the lead screw includes a second magnet;
wherein the second end of the lead screw is threadingly coupled with the rod such that, upon rotation of the first magnet due to an applied magnetic field, the lead screw and the second magnet rotate thereby causing the rod to displace relative to the housing, and
wherein the second magnet is axially fixed relative to the first magnet and the second magnet is configured to be monitored by an external sensor to determine a number of rotations of the lead screw.

2. The medical implant of claim 1, wherein a force can be determined by comparing an amount of rotation of the lead screw to an amount of rotation of the applied magnetic field, wherein the force is selected from the group consisting of a compression force, a distraction force, a tensile force and a rotation force.

3. The medical implant of claim 2, wherein the force comprises an amount of force applied to the first location and the second location within the body of the patient.

4. The medical implant of claim 2, wherein the force is derived at least in part from a magnetic coupling torque applied to the first magnet by the applied magnetic field.

5. The medical implant of claim 1, wherein the applied magnetic field is provided by an external adjustment device that is positioned external to the body of the patient.

6. The medical implant of claim 5, wherein the external adjustment device comprises an array of hall-effect sensors electronically tethered to the external adjustment device and configured to determine the amount of rotation of the lead screw.

7. A medical implant comprising:
a rod configured to be coupled to a first location within a body of a patient;
a hollow housing configured to be coupled to a second location within the body of the patient and having a first magnet disposed therein, the first magnet configured to revolve around an axis, wherein the hollow housing is configured to telescopically receive the rod; and
a lead screw having a first end and a second end opposing the first end, the second end of the lead screw being fixed relative to the first end of the lead screw, wherein the first end of the lead screw is mechanically coupled to the first magnet and the second end of the lead screw includes a second magnet;
wherein the second end of the lead screw is threadingly coupled with the rod such that, upon rotation of the first magnet due to an applied magnetic field, the lead screw and the second magnet rotate thereby causing the rod to displace relative to the hollow housing,
wherein the second magnet is axially fixed relative to the first magnet and the second magnet is configured to be monitored by an external sensor to determine a number of rotations of the lead screw, and
wherein movement of the rod relative to the hollow housing a dimension of the medical implant.

8. The medical implant of claim 7, wherein a force can be determined by comparing an amount of adjustment of the medical implant to an amount of rotation of the applied magnetic field, wherein the force is selected from the group consisting of a compression force, a distraction force, a tensile force and a rotation force.

9. The medical implant of claim 8, wherein the force comprises an amount of force applied to the first location and the second location within the body of the patient.

10. The medical implant of claim 8, wherein the force is derived at least in part from a magnetic coupling torque applied to the internal permanent magnet by the rotating magnetic field.

11. The medical implant of claim 7, wherein the applied magnetic field is provided by an external adjustment device that is positioned external to the body of the patient.

12. A medical implant system configured to adjust a dimension within a body of a patient, the medical implant system comprising:
a medical implant including:
a rod configured to be coupled to a first location within the body of the patient;
a housing configured to be coupled to a second location within the body of the patient and having a first magnet disposed therein, the first magnet configured to revolve around an axis, wherein the housing is configured to telescopically receive the rod; and
a lead screw having a first end and a second end opposing the first end, the second end of the lead screw being fixed relative to the first end of the lead screw, wherein the first end of the lead screw is mechanically coupled to the first magnet and the second end of the lead screw includes a second magnet; and
an external adjustment device configured to apply a magnetic field to the medical implant,
wherein the second end of the lead screw is threadingly coupled with the rod such that, upon rotation of the first magnet due to the applied magnetic field from the external adjustment device, the lead screw and the second magnet rotate thereby causing the rod to displace relative to the housing, and
wherein the second magnet is axially fixed relative to the first magnet and the second magnet is configured to be monitored by an external sensor to determine a number of rotations of the lead screw.

13. The medical implant system of claim 12, wherein a force can be determined by comparing an amount of rotation of the lead screw to an amount of rotation of the applied magnetic field, wherein the force is selected from the group consisting of a compression force, a distraction force, a tensile force and a rotation force.

14. The medical implant system of claim 13, wherein the force comprises an amount of force applied to the first location and the second location within the body of the patient.

15. The medical implant system of claim 13, wherein the force is derived at least in part from a magnetic coupling torque applied to the first magnet by the applied magnetic field.

16. The medical implant system of claim 12, wherein the external adjustment device is positioned external to the body of the patient.

17. The medical implant system of claim 12, wherein the external adjustment device comprises an array of hall-effect sensors electronically tethered to the external adjustment device and configured to determine the amount of rotation of the lead screw.
